(19) United States
(12) Patent Application Publication
(10) Pub. No.: US 2026/0071268 A1
(43) Pub. Date: Mar. 12, 2026

(54) METHODS OF INDUCING SITE-SPECIFIC CLEAVAGE OF RNA POLYNUCLEOTIDES WITH T2 FAMILY RIBONUCLEASES

(71) Applicant: Waters Technologies Corporation, Milford, MA (US)

(72) Inventors: Balasubrahmanyam Addepalli, Bellingham, MA (US); Tatiana Johnston, Barrington, RI (US); Matthew A. Lauber, North Smithfield, RI (US)

(21) Appl. No.: 19/321,639

(22) Filed: Sep. 8, 2025

Related U.S. Application Data
(60) Provisional application No. 63/692,272, filed on Sep. 9, 2024.

(51) Int. Cl. C12Q 1/6872; C12N 9/22; C12N 15/10

(52) U.S. Cl. CPC C12Q 1/6872; C12N 9/22; C12N 15/10

(57) ABSTRACT

Disclosed herein are methods that provide optimal conditions for site-specific digestion and mapping of the sequence of RNA polynucleotides using T2 family endoribonucleases, such as RNase MC1 and Cusativin.

### CROSS-REFERENCE TO RELATED APPLICATIONS

This application claims the benefit of and priority to U.S. Provisional Patent Application No. 63/692,272, filed Sep. 9, 2024, the entire contents of which are incorporated by reference herein.

### SEQUENCE LISTING

This application contains a Sequence Listing which has been submitted electronically in XML format and is hereby incorporated by reference in its entirety. Said XML copy, created on Sep. 3, 2025, is named “WAC-445US_SL.xml” and is 39,667 bytes in size.

### BACKGROUND

Enzymatic cleavage of RNA can be used to characterize the sequence of RNA polynucleotides. Complete hydrolysis of RNA leads to loss of location-specific information of nitrogenous bases. Therefore, limited digestion is typically performed to partially break down RNA, yielding a set of oligonucleotides for subsequent separation and analysis by liquid chromatography-mass spectrometry (LC-MS). Specific endoribonucleases, such as RNase T1, are sometimes used to generate a fingerprint of oligonucleotide fragments from which the sequence and position of specific nucleotides are ascertained. However, these commercially available enzymes typically yield smaller (<5-mers) digestion products, depending on the enzyme-recognized nucleotide frequency in the target sequence. This leads to incomplete sequence coverage and low-confidence data. The shortcomings of such methods are especially problematic when digesting longer RNA molecules, such as mRNA.

Enzymes with complementary specificity, such as RNase MCI and Cusativin, have been developed in an attempt to address these problems. However, their use for RNA mapping has been unsatisfactory due to lack of optimization and low reproducibility of oligonucleotide profiles for characterization of RNA sequences. RNase MCI and Cusativin belong to T2 family of Ribonucleases. Members of T2 family are endoribonucleases that cleave RNA in a non-specific fashion. Although RNase MCI and Cusativin can exhibit some nucleotide preference when cleaving RNA, their specificity is poorly defined under suboptimal conditions, thereby requiring cumbersome steps for LC-MS peak identification and oligonucleotide characterization.

### SUMMARY OF THE DISCLOSURE

Disclosed herein are methods that provide optimal conditions for site-specific digestion and mapping of the sequence of RNA polynucleotides using T2 family endoribonucleases (e.g., RNase MC1 and Cusativin).

Disclosed herein, in certain embodiments, is a method of inducing site-specific cleavage of one or more RNA polynucleotides, comprising contacting the one or more RNA polynucleotides with an RNase MC1 (MC1) enzyme in a solution having a pH of 6.5-9.5 (e.g., 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5). In certain embodiments, the solution has a pH of 8.0. In certain embodiments, the solution has a pH of 9.0.

In certain embodiments, the solution comprises the MC1 enzyme at a concentration of 10-500 U (e.g., 10-500, 20-480, 30-460, 40-420, 50-400, 60-380, 70-360, 80-340, 100-320, 120-300, 140-280, 160-260, 180-240, or 200-220 U).

In certain embodiments, the MC1 enzyme exhibits cleavage specificity for a dinucleotide pair selected from the group consisting of: A-U, C-U, U-U, C-A, and C-G.

In certain embodiments, the site-specific cleavage of the one or more RNA polynucleotides is improved by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to site-specific cleavage of the one or more RNA polynucleotides in a solution having a pH less than 6.5.

In certain embodiments, the method increases production of unique RNA polynucleotides by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to the production of unique RNA polynucleotides in a solution having a pH less than 6.5.

In certain embodiments, the MC1 enzyme has an amino acid sequence of SEQ ID NO: 1.

Disclosed herein, in certain embodiments, is a method of inducing site-specific cleavage of one or more RNA polynucleotides, comprising contacting the one or more RNA polynucleotides with a Cusativin enzyme in a solution having a pH of 8.5-9.5 (e.g., 8.5, 8.6, 8.7, 8.8, 8.9, 9.0, 9.1, 9.2, 9.3, 9.4, or 9.5). In certain embodiments, the solution has a pH of 9. In certain embodiments, the solution comprises the Cusativin enzyme at a concentration of 10-500 U (e.g., 10-500, 20-480, 30-460, 40-420, 50-400, 60-380, 70-360, 80-340, 100-320, 120-300, 140-280, 160-260, 180-240, or 200-220 U).

In certain embodiments, the Cusativin enzyme exhibits cleavage specificity for a dinucleotide pair selected from the group consisting of: C-A, C-G, C-U, U-A, U-U, A-U, and G-U.

In certain embodiments, the site-specific cleavage of the one or more RNA polynucleotides is improved by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to site-specific cleavage of the one or more RNA polynucleotides in a solution having a pH less than 6.5.

In certain embodiments, the method increases production of unique RNA polynucleotides by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to the production of unique RNA polynucleotides in a solution having a pH less than 6.5.

In certain embodiments, the Cusativin enzyme has an amino acid sequence of SEQ ID NO: 2.

In certain embodiments, the method further comprises the step of inactivating the enzyme (e.g., MC1 or Cusativin) at a temperature of 70° C.-80° C. (e.g., 70° C., 71° C., 72° C., 73° C., 74° C., 75° C., 76° C., 77° C., 78° C., 79° C., or 80° C.).

In certain embodiments, the method produces one or more RNA digestion products having a length of at least 3 (e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, or more) nucleotides. In certain embodiments, the method produces one or more RNA digestion products having a length of between 3 and 30 (e.g., 30, 29, 28, 27, 26, 25, 24, 23, 22, 21, 20, 19, 18, 17, 16, 15, 14, 13, 12, 11, 10, 9, 8, 7, 6, 5, 4, or 3) nucleotides from an RNA polynucleotide having a length of 30-100 nucleotides.

In certain embodiments, the one or more RNA polynucleotides is an RNA oligonucleotide, single guide RNA (sgRNA) polynucleotide, mRNA polynucleotide, microRNA polynucleotide, small nuclear RNA (snRNA) polynucleotide, transfer RNA (tRNA) polynucleotide, small interfering RNA (siRNA) polynucleotide, small nucleolar RNA (snoRNA) polynucleotide, ribosomal RNA (rRNA) polynucleotide, long noncoding RNA (lncRNA) polynucleotide, or Piwi-interacting RNA polynucleotide.

In certain embodiments, the method is used in conjunction with a method for sequencing the one or more RNA polynucleotides. In certain embodiments, the method is used in conjunction with liquid chromatography-mass spectroscopy (LC-MS) analysis of the one or more RNA polynucleotides. In certain embodiments, the LC-MS is ion pairing reversed phase LC-MS (IP-RP-LC-MS).

In certain embodiments, the solution further comprises ethylenediaminetetraacetic acid (EDTA) at a concentration that is between 2 mM and 5 mM. In certain embodiments, the EDTA concentration is 2 mM, 3 mM, 4 mM, or 5 mM. In certain embodiments, the solution further comprises EDTA at a concentration that is between 10 mM and 15 mM. In certain embodiments, the EDTA concentration is 10 mM, 11 mM, 12 mM, 13 mM, 14 mM, or 15 mM.

In certain embodiments, the site-specific cleavage of the one or more RNA polynucleotides is improved by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to site-specific cleavage of the one or more RNA polynucleotides in a solution having an EDTA concentration less than 2 mM.

In certain embodiments, the site-specific cleavage of the one or more RNA polynucleotides is improved by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to site-specific cleavage of the one or more RNA polynucleotides in a solution having an EDTA concentration less than 10 mM.

In certain embodiments, the method increases production of unique RNA polynucleotides by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to the production of unique RNA polynucleotides in a solution having an EDTA concentration less than 2 mM.

In certain embodiments, the method increases production of unique RNA polynucleotides by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to the production of unique RNA polynucleotides in a solution having an EDTA concentration less than 10 mM.

Disclosed herein, in certain embodiments, is a method of determining the nucleic acid sequence of an RNA polynucleotide of interest, comprising: (a) contacting the RNA polynucleotide of interest with an MCI enzyme in a solution having a pH of 6.5-9.5 (e.g., 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5) to produce a population of RNA oligonucleotides, wherein at least two RNA oligonucleotides within the population have a nucleic acid sequence overlap of at least 1 nucleotide; (b) subjecting the RNA oligonucleotides to LC-MS analysis to determine the nucleic acid sequence of each of the RNA oligonucleotides within the population; and (c) aligning the nucleic acid sequences of the plurality of RNA oligonucleotides using the overlap between the at least two RNA oligonucleotides, thereby determining the nucleic acid sequence of the RNA polynucleotide of interest. In certain embodiments, the solution has a pH of 8.0. In certain embodiments, the solution has a pH of 9.0.

Disclosed herein, in certain embodiments, is a method of determining the nucleic acid sequence of an RNA polynucleotide of interest, comprising: (a) contacting the RNA polynucleotide of interest with a Cusativin in a solution having a pH of 6.5-9.5 (e.g., 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5) to produce a population of RNA oligonucleotides, wherein at least two RNA oligonucleotides within the population have a nucleic acid sequence overlap of at least 1 nucleotide; (b) subjecting the RNA oligonucleotides to LC-MS analysis to determine the nucleic acid sequence of each of the RNA oligonucleotides within the population; and (c) aligning the nucleic acid sequences of the plurality of RNA oligonucleotides using the overlap between the at least two RNA oligonucleotides, thereby determining the nucleic acid sequence of the RNA polynucleotide of interest. In certain embodiments, the solution has a pH of 9.0.

In certain embodiments, the nucleic acid sequence of each RNA oligonucleotide within the population is determined using a unique mass tag of each ribonucleotide within the RNA oligonucleotide. In certain embodiments, the at least two RNA oligonucleotides within the population are not identical. In certain embodiments, step (a) further comprises a step of inactivating the enzyme at a temperature of at least 70° C.

### Definitions

Unless defined otherwise, all technical and scientific terms used herein have the same meaning as is commonly understood by one of skill in the art to which the claimed subject matter pertains. Generally, nomenclatures utilized in connection with and techniques of molecular biology, RNA chemistry, and protein chemistry described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a,” “and,” and “the” include plural referents unless the context clearly indicates otherwise. Thus, e.g., reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, e.g., reference to a range of 90-100%, includes 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1-, 2-, 3-, 4-, 5-, 6-, 7-, 8-, 9-, 10-, 11-, 12-, 13-, 14-, 15-, 16-, 17-, 18-, 19-, 20-fold, etc., as well as 1.1-, 1.2-, 1.3-, 1.4-, 1.5-fold, etc., 2.1-, 2.2-, 2.3-, 2.4-, 2.5-fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, the term “digestion products” refers to one or more RNA fragments produced from enzymatic cleavage of an RNA polynucleotide with an endoribonuclease (e.g., RNase MC1 or Cusativin). In certain embodiments, a “digestion product” is an RNA oligonucleotide.

As used herein, the term “induce” refers to a means or method for initiating or increasing a particular activity (e.g., site-specific enzymatic cleavage of RNA, e.g., with a T2 family endoribonuclease). In certain embodiments, “inducing” site-specific cleavage of an RNA polynucleotide by a T2 family endoribonuclease (e.g., RNase MC1 or Cusativin) means making fewer but more site-selective cleavages of a target RNA, e.g., to produce longer RNA digestion products. Therefore, “inducing” an activity of an enzyme does not necessarily imply causing increased activity of an enzyme, but can mean shifting the operational regime of an enzyme into a particular mode to achieve a desired result (e.g., fewer and more site-selective hydrolytic cleavages of a target RNA by an endoribonuclease). In certain embodiments, inducing the activity means that there was no activity prior to the induction and some (low, intermediate, or high) activity thereafter. In certain embodiments, inducing the activity means that there was some activity prior to the induction and increased activity thereafter. In certain embodiments, the activity following induction is increased by 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to the activity prior to the induction.

The terms “oligonucleotide” and “polynucleotide” as used herein, are defined as it is generally understood by the skilled person as a molecule including two or more covalently linked nucleosides. Such covalently bound nucleosides may also be referred to as nucleic acid molecules or oligomers. Oligonucleotides are commonly made in the laboratory by enzymatic digestion or solid-phase chemical synthesis followed by purification. When referring to a sequence of the oligonucleotide, reference is made to the sequence or order of nucleobase moieties, or modifications thereof, of the covalently linked nucleotides or nucleosides.

As used herein, the terms “ribonucleic acid(s)” and “RNA” refer to oligoribonucleotides or polyribonucleotides as well as analogs of RNA, for example, made from nucleotide analogs. The RNA will typically have a base moiety of adenine (A), guanine (G), cytosine (C) and uracil (U), a sugar moiety of a ribose and a phosphate moiety of phosphate bonds. RNA molecules include both natural RNA and artificial RNA analogs. The RNA can be synthetic or can be isolated from a particular biological sample using any number of procedures which are well-known, wherein the particular chosen procedure is appropriate for the particular biological sample. RNA samples can include, for example, an RNA oligonucleotide, single guide RNA (sgRNA) polynucleotide, mRNA polynucleotide, microRNA polynucleotide, small nuclear RNA (snRNA) polynucleotide, transfer RNA (tRNA) polynucleotide, small interfering RNA (siRNA) polynucleotide, small nucleolar RNA (snoRNA) polynucleotide, ribosomal RNA (rRNA) polynucleotide, long noncoding RNA (lncRNA) polynucleotide, and/or Piwi-interacting RNA polynucleotide. No limitations are imposed on the base length of RNA. The LC-MS-based sequencing methods disclosed herein facilitate the sequencing of not only purified RNA samples, but also more complicated RNA samples containing mixtures of different RNAs.

As used herein, the terms “site-specific cleavage,” “cleavage specificity,” or the like refer to a property of an endoribonuclease, such as a T2 family endoribonuclease (e.g., RNase MC1 or Cusativin), characterized by a preference for cleavage of specific nucleotide positions or patterns of nucleotides (e.g., preference for specific dinucleotide pairs). For example, in the context of the T2 family endoribonucleases which are suitable for use with the disclosed methods, “site-specific cleavage” refers to the preference of these enzymes to target particular dinucleotide pairs within a target RNA sequence. In certain embodiments, an RNase MC1 enzyme exhibits preferential cleavage of an RNA polynucleotide of interest at a dinucleotide pair selected from the group consisting of: A-U, C-U, U-U, C-A, and C-G. In certain embodiments, a Cusativin enzyme exhibits preferential cleavage of an RNA polynucleotide of interest at a dinucleotide pair selected from the group consisting of: C-A, C-G, C-U, U-A, U-U, A-U, and G-U. In certain embodiments, “site-specific cleavage” means that an endoribonuclease may exhibit a weak preference for cleavage at certain nucleotide positions or dinucleotide pairs and a stronger preference for others. In certain embodiments, “site-specific cleavage” means that an endoribonuclease may exhibit no cleavage at certain nucleotide positions or dinucleotide pairs and some (e.g., low, intermediate, or high) cleavage at nucleotides or dinucleotide pairs. As is disclosed herein, such site-specific cleavage may be modulated by certain conditions, such as pH or presence of an additive (e.g., EDTA content).

### BRIEF DESCRIPTION OF THE DRAWINGS

FIGS. 1A-1C are chromatograms and illustrations showing RNase MC1-mediated RNA digestion of a 35-mer RNA polynucleotide at pH 7 (FIG. 1A) and pH 8 (FIG. 1B). “TIC”=total ion current. FIG. 1C shows an overlay of detected oligonucleotide digestion products to the nucleotide sequence of a 35-mer (SEQ ID NO: 39). Longer digestion products (SEQ ID NOS 6-9, respectively, in order of appearance) observed with high pH digestion are shown in bold font and italicized. All the digestion products indicated in the overlays contained a 3′ cyclic phosphate (cP).

FIGS. 2A-2B are chromatograms showing chromatograms and illustrations showing RNase MC1-mediated RNA digestion of a single guide RNA (sgRNA) targeting Hypoxanthinc Phosphoribosyltransferase (HPRT) RNA polynucleotide at pH 7 (FIG. 2A) and pH 8 (FIG. 2B).

FIGS. 3A-3E are chromatograms and illustrations showing RNase MC1-mediated RNA digestion of a 35-mer RNA polynucleotide in the absence of ethylenediaminetetraacetic acid (EDTA) or with varying concentrations of EDTA. FIGS. 3A-3D show ion chromatograms of 35-mer RNA digests performed without and with EDTA. (FIG. 3A) No EDTA; (FIG. 3B) 2 mM, (FIG. 3C) 5 mM, and (FIG. 3D) 10 mM EDTA. FIG. 3E shows an overlay of detected oligonucleotide digestion products mapped to the nucleotide sequence of the 35-mer RNA polynucleotide (SEQ ID NO: 39). Longer digestion fragments (SEQ ID NOS 6-9, respectively, in order of appearance) observed with high pH digestion are shown in bold font and italicized. Predominant sets of oligonucleotides detected in the absence and presence of EDTA are shown. Peaks were numbered based on their elution time. All the digestion products indicated in the overlays contained a 3′ cyclic phosphate (cP).

FIGS. 4A-4C are chromatograms showing RNase MC1-mediated RNA digestion of an HPRT sgRNA without EDTA or with varying concentrations of EDTA. (FIG. 4A) No EDTA; (FIG. 4B) 2 mM; and (FIG. 4C) 5 mM.

FIG. 5 shows plots illustrating mapping of HPRT sgRNA with liquid chromatography-ultraviolet-mass spectroscopy (LC-UV-MS) following digestion with RNase MC1 at pH 8. Top panel shows a total ion chromatogram of identified RNase-MC1-produced digestion products at pH 8. Bottom panel shows a UV trace of RNA digestion products. 44 digestion products of varying lengths and retention times were scored and annotated on the chromatogram.

FIG. 6 shows an overlay of identified RNase MC1-generated digestion products (SEQ ID NOS 14, 11, 15, 12, 20, 16 and 21, respectively, in order of appearance) (i.e., RNA oligonucleotide sequences) mapped over the full-length of the HPRT sgRNA sequence (SEQ ID NO: 10). All the digestion products indicated in the overlays contained a 3′ cyclic phosphate (cP).

FIGS. 7A-7C are chromatograms showing digestion products of HPRT sgRNA produced by RNase MCI without EDTA at pH 8 (FIG. 7A) or in the presence of 2 mM EDTA at pH 8 (FIG. 7B). FIG. 7C shows a chromatogram of undigested sgRNA as a control where the RNA was treated without enzyme under identical digestion conditions (no EDTA).

FIGS. 8A-8D are chromatograms showing modulation of RNase MC1-mediated digestion of HPRT sgRNA under high temperature. The reaction mixture was subjected to no heat exposure (FIG. 8A), 50° C. (FIG. 8B), 60° C. (FIG. 8C), 70° C. (FIG. 8D) for 15 min for subsequent digestion of sgRNA at 30° C. FIG. 8E is a bar chart showing the peak area of sgRNA incubated with no MCI enzyme or with MCI enzyme exposed at different temperatures.

FIGS. 9A-9B are chromatograms showing Cusativin-mediated RNA digestion of a 35-mer RNA polynucleotide at pH 7 (FIG. 9A) and pH 8 (FIG. 9B).

FIGS. 10A-10E are chromatograms and illustrations showing Cusativin-mediated RNA digestion of a 35-mer RNA polynucleotide in the absence of EDTA or with varying concentrations of EDTA. (FIG. 10A) No EDTA; (FIG. 10B) 10 mM; (FIG. 10C) 15 mM; and (FIG. 10D) 25 mM EDTA. FIG. 10E shows an overlay of detected oligonucleotide digestion products (SEQ ID NOS 6-9, respectively, in order of appearance) mapped to the nucleotide sequence of a 35-mer RNA polynucleotide (SEQ ID NO: 39). Predominant sets of oligonucleotides detected in the absence and presence of EDTA are shown. All the digestion products indicated in the overlays contained a 3′ cyclic phosphate (cP).

FIGS. 11A-11D are chromatograms showing Cusativin-mediated RNA-digestion products of HPRT sgRNA without EDTA or with varying concentrations of EDTA. (FIG. 11A) No EDTA; (FIG. 11B) 10 mM; (FIG. 11C) 15 mM; and (FIG. 11D) Undigested sgRNA (treated without the enzyme under identical digestion conditions, i.e., no EDTA). Peaks beyond 8.56 minutes represent the longer digestion products.

FIG. 12 shows plots illustrating mapping of HPRT sgRNA with LC-UV-MS following digestion with Cusativin at pH 9. Top panel shows a total ion chromatogram of identified Cusativin-produced digestion products. Bottom panel shows a UV trace of oligonucleotide digestion products.

FIG. 13 shows an overlay of identified Cusativin-produced digestion products (SEQ ID NOS 32, 33, 30, 31 and 34, respectively, in order of appearance) (i.e., RNA oligonucleotide sequences) mapped over the full-length HPRT sgRNA sequence (SEQ ID NO: 10). All the digestion products indicated in the overlays contained a 3′ cyclic phosphate (cP). Overlapping features of the oligonucleotides facilitate high-confidence oligonucleotide mapping.

FIGS. 14A-14E are chromatograms showing modulation of Cusativin-mediated digestion of HPRT sgRNA under high temperature. The reaction mixture was subjected to no heat exposure (FIG. 14A), 50° C. (FIG. 14B), 60° C. (FIG. 14C), or 70° C. (FIG. 14D) for 15 min for subsequent digestion of sgRNA at 30° C. FIG. 14E shows an undigested sgRNA under identical conditions except that no enzyme was added. FIG. 14F is a bar chart showing the peak area of sgRNA incubated with no Cusativin enzyme or with Cusativin enzyme exposed at different temperatures.

FIGS. 15A-15C are chromatograms showing chromatograms and illustrations showing RNase MC1-mediated RNA digestion of a HPRT-targeting sgRNA at pH 6.8 (FIG. 15A), pH 8 (FIG. 15B), and pH 9 (FIG. 15C).

FIGS. 16A-16C are chromatograms showing chromatograms and illustrations showing RNase Cusativin-mediated RNA digestion of a HPRT-targeting sgRNA at pH 6.8 (FIG. 16A), pH 8 (FIG. 16B), and pH 9 (FIG. 16C).

### DETAILED DESCRIPTION

Disclosed herein are methods providing optimal RNA digestion conditions required by T2 family endoribonucleases, such as RNase MC1 (“MC1”) and Cusativin, to achieve site-specific cleavage of a target RNA polynucleotide. Such methods can be used, in certain embodiments, for efficient RNA mapping analysis, e.g., using liquid chromatograph-mass spectrometry (LC-MS). The present disclosure demonstrates that cleavage specificity of highly active MCI and Cusativin enzymes is controlled by at least two factors, including pH and additive concentration (e.g., ethylenediaminetetraacetic acid (EDTA) concentration). Under high pH, these enzymes generate predictable cleavage of phosphodiester bonds in a dinucleotide-specific manner. At pH 8-9, MCI is shown herein to cleave RNA polynucleotide pairs A-U, C-U, U-U, C-A, and C-G. The extent of cleavage is determined by the RNA sequence cleavage site distribution. Unlike RNase TI, which generates shorter products from G-rich regions, MCI is shown to skip one or more cleavage sites when they are positioned in tandem, thus generating longer digestion products to provide greater sequence coverage of an RNA polynucleotide of interest. Similar cleavage behavior is observed herein when pH 9 was used to digest RNA polynucleotides with Cusativin. Cusativin exhibits cleavage specificity at dinucleotide pairs C-A, C-G, C-U, U-A, U-U, A-U, and G-U under high pH conditions.

Furthermore, introduction of an additive (e.g., EDTA; 2-5 mM for MC1 and 10-15 mM for Cusativin) in the digestion mix resulted in skipping of cleavage sites and generation of longer oligonucleotide digestion products. Under suboptimal pH conditions or in the absence of EDTA, non-specific cleavage by these enzymes resulted in suboptimal oligonucleotide product generation and lower sequence coverage of the RNA polynucleotide of interest.

The consistency of this cleavage behavior improved the predictability, reproducibility, and confident oligonucleotide assignment during data analysis. Longer digestion products have two important advantages: (1) sequence overlaps at 5′ and 3′ ends of longer digestion products facilitate sequence reconstruction; and (2) increased number of unique RNA digestion products that map to specific regions of the RNA sequence act as a sequence fingerprint and improves confidence in data interpretation.

Moreover, the present disclosure demonstrates that these T2 endoribonucleases can be inactivated by heat exposure (e.g., a temperature of at least 70° C.), unlike other endoribonucleases that require RNase inhibitors or immobilization on beads to avoid excessive cleavage, thereby enhancing reproducibility of RNA digestion and sequence mapping.

### Endoribonuclease-Mediated RNA Mapping

The emergence and growth of RNA-based therapeutics has created an unmet need for the development of improved methods for analysis and characterization of RNA (e.g. mRNA). Traditionally, RNA-sequencing technologies primarily relied on polymerization-dependent copying of RNA into deoxyribonucleotides through Watson-Crick base pairing. However, LC-MS has emerged as an effective method for rapid, high-throughput characterization of RNA. Base-specific RNase digestion of RNA polynucleotides followed by separation and MS using ion-pairing, reverse phase liquid chromatography, or IP-RP-LC-MS facilitates mapping of RNA sequences with high coverage.

One limitation of this approach is the dearth of commercially available, nucleotide-specific endoribonucleases (RNases). Guanosine-specific RNase Tl and pyrimidine-selective RNase A are both commercially available and compatible with MS-based RNA sequence mapping. Purine-selective RNase U2 is also commercially available, but only sparingly so. However, optimal RNA mapping requires generation of sufficient overlapping digestion products from multiple RNases to reduce redundancies in digestion product sequences and improve sequence coverage.

Alternative approaches for producing overlapping RNA digestion products are available. These include partial RNase-mediated digestion, use of non-specific nucleases, and alkaline hydrolysis. However, these strategies also suffer from certain shortcomings, including non-specific digestion and sub-optimal reaction conditions that cause excessive or insufficient digestion. These limitations lead to poor analytical reproducibility and labor-intensive optimization processes. Therefore, new methods for RNase-mediated RNA mapping with complementary nucleoside specificity could prove useful.

RNase MC1 is a member of the RNase T2 family first isolated from bitter gourd seeds and is known to exhibit uridine-specific cleavage of RNA. Further, cucumber seed-derived Cusativin is known to exhibit cytidine-specific cleavage of RNA. However, the conditions under which these RNases exhibit optimal digestion for RNA mapping analysis have not been determined.

Disclosed herein, in certain embodiments, are methods for improving RNA sequence mapping using LC-MS analysis in combination with RNA digestion using T2 family endoribonucleases that produce long digestion products, thereby facilitating more comprehensive sequence coverage and increasing reproducibility of data in RNA sequencing analysis. The method includes, in certain embodiments, (1) digesting an RNA of interest with a T2 family endoribonuclease (e.g., MC1 or Cusativin) under conditions optimal for site-specific cleavage of the RNA by the endoribonuclease to generate digestion products comprising oligonucleotide fragments of the target RNA sequence; and (2) analyzing the digestion products using an analytical method to characterize sequences of oligonucleotide fragments of the RNA and reconstruct the partial or full sequence of the RNA of interest. In certain embodiments, the endoribonuclease is an MCI enzyme. In certain embodiments, the endoribonuclease is Cusativin enzyme. In certain embodiments, the analytical method includes LC-MS.

In certain embodiments, a method disclosed herein includes inducing site-specific cleavage of one or more (e.g., 1, 2, 3, 4, 5, 6, 7, 8, 9, 10, or more) RNA polynucleotides, the method comprising contacting the one or more RNA polynucleotides with an MC1 enzyme in a solution having a pH of 6.5-9.5 (e.g., 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5). In certain embodiments, the solution has a pH of 8. In certain embodiments, the solution further comprises ethylenediaminetetraacetic acid (EDTA) at a concentration that is between 2 mM and 5 mM (e.g., 2 mM, 3 mM, 4 mM, or 5 mM).

In certain embodiments, a method disclosed herein includes inducing site-specific cleavage of one or more (e.g., 1, 2, 3, 4, 5, 6, 7, 8, 9, 10, or more) RNA polynucleotides, comprising contacting the one or more RNA polynucleotides with an MC1 enzyme in a solution comprising EDTA at a concentration that is between 2 mM and 5 mM (e.g., 2 mM, 3 mM, 4 mM, or 5 mM). In certain embodiments, the solution has a pH of 6.5-9.5 (e.g., 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5). In certain embodiments, the solution has a pH of 8. In certain embodiments, the solution has a pH of 9.

In certain embodiments, a method disclosed herein includes inducing site-specific cleavage of one or more (e.g., 1, 2, 3, 4, 5, 6, 7, 8, 9, 10, or more) RNA polynucleotides, the method comprising contacting the one or more RNA polynucleotides with a Cusativin enzyme in a solution having a pH of 6.5-9.5 (e.g., 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5). In certain embodiments, the solution has a pH of 9. In certain embodiments, the solution further comprises EDTA at a concentration that is between 10 mM and 15 mM (e.g., 10 mM, 11 mM, 12 mM, 13 mM, 14 mM, or 15 mM).

In certain embodiments, a method disclosed herein includes inducing site-specific cleavage of one or more (e.g., 1, 2, 3, 4, 5, 6, 7, 8, 9, 10, or more) RNA polynucleotides, the method comprising contacting the one or more RNA polynucleotides with a Cusativin enzyme in a solution comprising a concentration of EDTA that is between 10 mM and 15 mM (e.g., 10 mM, 11 mM, 12 mM, 13 mM, 14 mM, or 15 mM). In certain embodiments, the solution has a pH of 6.5-9.5 (e.g., 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5). In certain embodiments, the solution has a pH of 9.

Also disclosed herein are methods for terminating an MC1-mediated or Cusativin-mediated RNA digestion reaction using a heat inactivation step. In certain embodiments, the heat inactivation step comprises inactivating the MC1 enzyme or the Cusativin enzyme at a temperature of at least 70° C. (e.g., at least 70° C., 71° C., 72° C., 73° C., 74° C., 75° C., 76° C., 77° C., 78° C., 79° C., 80° C., or more). In certain embodiments, the heat inactivation step comprises inactivating the MC1 enzyme or the Cusativin enzyme at a temperature between 70° C. and 80° C. (e.g., 70° C., 71° C., 72° C., 73° C., 74° C., 75° C., 76° C., 77° C., 78° C., 79° C., or 80° C.).

Concentrations of T2 family endoribonucleases (e.g., MC1 or Cusativin) suitable for use with the RNA digestion methods disclosed herein may vary depending on various factors, including the size (i.e., length) of the RNA polynucleotide of interest, the starting quantity of RNA polynucleotides in the reaction, the desired quantity of digestion products, and the composition of the digestion reaction mix, among others. In certain embodiments, a reaction mixture (i.e., a solution comprising one or more RNA polynucleotides of interest and a T2 family endoribonuclease) comprises an MC1 enzyme at a concentration of 10-500 U (where 1 U produces a change in A260 of 0.01 a.u./min in a 0.1 mL reaction at pH 5.6 and 30° C. using 1 mg/mL yeast tRNA as a substrate). In certain embodiments, a reaction mixture comprises a Cusativin enzyme at a concentration of 10-500 U.

In certain embodiments, the MC1 enzyme cleaves one or more RNA polynucleotides of interest at a dinucleotide pair selected from the group consisting of: A-U, C-U, U-U, C-A, and C-G. In certain embodiments, the Cusativin enzyme cleaves one or more RNA polynucleotides of interest at a dinucleotide pair selected from the group consisting of: C-A, C-G, C-U, U-A, U-U, A-U, and G-U.

The disclosed methods allow for tuning of reaction conditions to achieve a desired distribution of digestion product sizes, thereby increasing the number of unique digestion products, enhancing sequence coverage, and improving reproducibility of RNA sequence mapping data. In certain embodiments, the reaction conditions are optimized to produce one or more digestion products having a length of at least 3 (e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, or more) nucleotides. In certain embodiments, the reaction conditions are optimized to produce one or more digestion products having a length of 30 or less (e.g., 30, 29, 28, 27, 26, 25, 24, 23, 22, 21, 20, 19, 18, 17, 16, 15, 14, 13, 12, 11, 10, 9, 8, 7, 6, 5, 4, or 3) nucleotides, depending on the length of the substrate RNA polynucleotide (e.g., from a target RNA polynucleotide having a length of 30-100 nucleotides).

RNA molecules suitable for analysis using a method disclosed herein include any one of known RNA types, including but not limited to RNA oligonucleotides, single guide RNA (sgRNA), mRNA, microRNA, small nuclear RNA (snRNA), transfer RNA (tRNA), small interfering RNA (siRNA), small nucleolar RNA (snoRNA), ribosomal RNA (rRNA), long noncoding RNA (lncRNA), or Piwi-interacting RNA. The size of an RNA polynucleotide(s) of interest suitable for use with the disclosed methods can vary. In certain embodiments, the one or more RNA polynucleotides suitable for analysis with the disclosed methods has a length that is between 35-100 nucleotides.

In certain embodiments, the methods disclosed herein improve site-specific cleavage by RNase MC1 of one or more RNA polynucleotides of interest, e.g., by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to site-specific cleavage of the one or more RNA polynucleotides in a solution having a pH less than 6.5. In certain embodiments, the methods disclosed herein improve site-specific cleavage by RNase MC1 of the one or more RNA polynucleotides, e.g., by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to site-specific cleavage of the one or more RNA polynucleotides in a solution having an EDTA concentration less than 2 mM.

In certain embodiments, the methods disclosed herein improve site-specific cleavage by Cusativin of one or more RNA polynucleotides of interest, e.g., by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to site-specific cleavage of the one or more RNA polynucleotides in a solution having a pH less than 6.5. In certain embodiments, the methods disclosed herein improve site-specific cleavage by Cusativin of the one or more RNA polynucleotides, e.g., by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to site-specific cleavage of the one or more RNA polynucleotides in a solution having an EDTA concentration less than 10 mM.

In certain embodiments, the methods disclosed herein increase production of one or more unique RNA polynucleotides by RNase MC1, e.g., by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to the production of unique RNA polynucleotides in a solution having a pH less than 6.5. In certain embodiments, the methods disclosed herein increase production of one or more unique RNA polynucleotides by RNase MC1, e.g., by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to the production of unique RNA polynucleotides in a solution having an EDTA concentration less than 2 mM.

In certain embodiments, the methods disclosed herein increase production of one or more unique RNA polynucleotides by Cusativin, e.g., by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to the production of unique RNA polynucleotides in a solution having a pH less than 6.5. In certain embodiments, the methods disclosed herein increase production of one or more unique RNA polynucleotides by Cusativin, e.g., by at least 5%, 10%, 15%, 20%, 25%, 30%, 35%, 40%, 45%, 50%, 60%, 70%, 80%, 90%, 100%, 120%, 140%, 160%, 180%, 200%, 250%, 300%, 350%, 400%, 450%, 500%, 600%, 700%, 800%, 900%, 1000%, or more, as compared to the production of unique RNA polynucleotides in a solution having an EDTA concentration less than 10 mM.

### Applications of Disclosed Methods

Methods disclosed herein can be advantageously used for a variety of applications in which site-specific digestion of RNA polynucleotides is required. In a nonlimiting example, the disclosed methods can be used to digest RNA polynucleotides into smaller oligonucleotide fragments for sequencing by way of LC-MS. The LC-MS-based RNA sequencing methods suitable for use with the methods disclosed herein advantageously facilitate sequencing of purified RNA samples, as well as samples containing multiple RNA species, including mixtures of RNA derived from a biological sample. This strategy can be applied to the de novo sequencing of RNA sequences carrying both canonical and structurally atypical nucleosides.

The sequence information of RNA polynucleotides can be determined using the methods disclosed herein in combination with, e.g., LC-MS methods. In one example, the RNA to be sequenced is subjected to controlled degradation using the disclosed methods. As used herein, the terms degradation, digestion, and cleavage may be used interchangeably. It is understood that the degradation, digestion, or cleavage, of RNA refers to breaks in the RNA strand resulting in fragmentation of the RNA into two or more fragments. In general, such fragmentation for purposes of the present disclosure is determined by the specificity of the T2 family endoribonuclease used for cleavage (e.g., MC1 or Cusativin). RNA's natural tendency to be degraded can be advantageously used to generate a sequence ladder, i.e., a mass latter, for subsequent sequence determination via, e.g., LC-MS. By controlling pH and additive (e.g., EDTA) concentration in the reaction mixture, cleavage along the target RNA molecule backbone may be achieved to produce long RNA oligonucleotide fragments, thus simplifying downstream MS data analysis and improving reproducibility. Additionally, the timing of exposure to the T2 family endoribonuclease can be controlled by terminating the hydrolysis reaction through heat inactivation of the endoribonuclease, as described herein. Once preparation of RNA oligonucleotide fragments using the disclosed methods is performed, the RNA fragments can be analyzed by any of a variety of means including LC-MS (e.g., reverse phase (RP)-LC-MS), collision-induced dissociation-mass spectrometry (CDS-MS) polyacrylamide gel electrophoresis (PAGE), high throughput sequencing (e.g., next generation sequencing) methods, among others.

Following digestion of RNA polynucleotides using the disclosed methods, LC-MS-based methods can be performed to identify sequences of RNA digestion products by converting LC-MS data into RNA sequence information. Computational algorithms that extract, align, and process relevant mass peaks from the mass spectrum can be used to fully or partially reconstruct the digested RNA polynucleotide sequence. These computational methods are routine and conventional. The unique mass tag of each ribonucleotide allows one to determine the primary nucleotide sequence of the RNA. Such methods will be of special valuable to those engaged in research, manufacture, and quality control of RNA-based therapeutics, as well as the regulatory entities.

### T2 Family Endoribonucleases

Endoribonuclease enzymes suitable for use with the methods disclosed herein include the T2 family endoribonucleases. In certain embodiments, the T2 family endoribonuclease is RNase MC1 (“MC1”). In certain embodiments, MC1 is a recombinant MC1 enzyme. In certain embodiments, MC1 is isolated from a natural product (e.g., bitter gourd seeds). In certain embodiments, MC1 has an amino acid sequence of SEQ ID NO: 1.

(SEQ ID NO: 1)
FDSFWFVQQWPPAVCSFQKSGSCPGSGLRTFTIHGLWPQQSGTSLTNCPG
SPFDITKISHLQSQLNTLWPNVLRANNQQFWSHEWTKHGTCSESTFNQAA
YFKLAVDMRNNYDIIGALRPHAAGPNGRTKSRQAIKGFLKAKFGKFPGLR
CRTDPQTKVSYLVQVVACFAQDGSTLIDCTRDTCGANFIF

In certain embodiments, the MC1 enzyme is encoded by a nucleic acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 2. In certain embodiments, the MC1 enzyme is encoded by a nucleic acid sequence having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 2. In certain embodiments, the MC1 enzyme is encoded by a nucleic acid sequence having at least 90% (e.g., at least 90%, 91%, 92%, 93%, 94%, 95%, 96%, 97%, 98%, 99%, or more) sequence identity to SEQ ID NO: 2. In certain embodiments, the MC1 enzyme is encoded by a nucleic acid sequence having at least 95% (e.g., at least 95%, 96%, 97%, 98%, 99%, or more) sequence identity to SEQ ID NO: 2. In certain embodiments, the MC1 enzyme is encoded by a nucleic acid sequence having at least 99% (e.g., at least 99%, or more) sequence identity to SEQ ID NO: 2. In certain embodiments, the MC1 enzyme is encoded by a nucleic acid sequence of SEQ ID NO: 2.

(SEQ ID NO: 2)
TTCGACTCCTTTTGGTTTGTTCAGCAGTGGCCGCCAGCAGTTTGCAGTTT
CCAAAAAAGTGGTAGTTGCCCTGGTAGTGGTCTGCGCACGTTTACTATCC
ATGGCCTGTGGCCGCAACAGAGCGGCACAAGCCTGACTAACTGTCCGGGT
TCTCCGTTTGATATCACCAAGATCAGCCACCTGCAGAGCCAGCTCAATAC
CCTGTGGCCGAACGTGCTGCGTGCCAATAACCAACAGTTCTGGAGTCACG
AGTGGACGAAACATGGCACATGCTCCGAAAGCACCTTTAACCAAGCGGCC
TACTTCAAACTTGCGGTCGACATGCGCAACAACTATGATATCATCGGGGC
CTTGCGTCCGCACGCAGCAGGCCCGAACGGCCGTACCAAATCACGTCAGG
CCATCAAAGGGTTTCTGAAAGCGAAATTTGGTAAATTTCCGGGCTTACGT
TGTCGTACCGATCCGCAAACGAAAGTTAGCTATCTGGTTCAAGTCGTCGC
ATGCTTCGCCCAGGATGGTTCAACCTTAATTGATTGTACCCGCGACACGT
GTGGCGCCAATTTCATCTTT

In certain embodiments, the T2 family endoribonuclease is Cusativin. In certain embodiments, Cusativin is a recombinant Cusativin enzyme. In certain embodiments, Cusativin is isolated from a natural product (e.g., cucumber seeds). In certain embodiments, Cusativin has an amino acid sequence of SEQ ID NO: 3.

(SEQ ID NO: 3)
MEKTKSVDVVFFVFVLTILFPIVKSQTFDDFWFVQQWPPAVCTLQSGRCV
GRGTRSFTIHGLWPQKGGRSVTNCTGNQFDFTKIAHLENDLNVVWPNVVT
GNNKFFWGHEWNKHGICSESKFDEAKYFQTAINMRHGIDLLSVLRTGGVG
PNGASKAKQRVETAISSHFGKDPILRCKKASNGQVLLTEIVMCFDDDGVT
LINCNKARSNCAGSFIF

In certain embodiments, the Cusativin enzyme is encoded by a nucleic acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 4. In certain embodiments, the Cusativin enzyme is encoded by a nucleic acid sequence having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 4. In certain embodiments, the Cusativin enzyme is encoded by a nucleic acid sequence having at least 90% (e.g., at least 90%, 91%, 92%, 93%, 94%, 95%, 96%, 97%, 98%, 99%, or more) sequence identity to SEQ ID NO: 4. In certain embodiments, the Cusativin enzyme is encoded by a nucleic acid sequence having at least 95% (e.g., at least 95%, 96%, 97%, 98%, 99%, or more) sequence identity to SEQ ID NO: 4. In certain embodiments, the Cusativin enzyme is encoded by a nucleic acid sequence having at least 99% (e.g., at least 99%, or more) sequence identity to SEQ ID NO: 4. In certain embodiments, the Cusativin enzyme is encoded by a nucleic acid sequence of SEQ ID NO: 4.

(SEQ ID NO: 4)
ATGGAAAAAACAAAGAGTGTCGATGTTGTGTTCTTCGTGTTTGTCTTAAC
CATTTTGTTTCCTATCGTTAAATCGCAAACTTTCGATGATTTTTGGTTTG
TTCAGCAATGGCCACCAGCTGTTTGTACTTTACAATCAGGACGATGTGTA
GGACGAGGGACTCGATCCTTCACAATTCATGGTTTGTGGCCTCAAAAGGG
AGGACGTTCTGTGACTAATTGTACTGGCAATCAATTTGATTTCACTAAGA
TTGCACATTTAGAAAACGATCTAAACGTAGTTTGGCCGAATGTGGTGACG
GGAAACAACAAATTCTTTTGGGGTCATGAATGGAACAAACATGGGATTTG
CTCAGAGAGCAAGTTTGACGAAGCGAAGTACTTCCAAACAGCGATAAACA
TGAGGCACGGTATCGACCTTCTTAGTGTGCTGAGAACTGGTGGAGTAGGA
CCAAATGGAGCCTCCAAGGCAAAGCAAAGAGTCGAAACCGCGATTTCATC
CCATTTTGGAAAAGATCCAATTCTTCGATGTAAAAAAGCATCAAACGGCC
AAGTCTTATTGACGGAGATTGTGATGTGCTTCGATGACGATGGTGTCACC
CTTATAAACTGTAACAAAGCAAGATCCAATTGTGCTGGGAGCTTTATTTT
T

In certain embodiments, the Cusativin enzyme is encoded by a nucleic acid sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 5. In certain embodiments, the Cusativin enzyme is encoded by a nucleic acid sequence having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 5. In certain embodiments, the Cusativin enzyme is encoded by a nucleic acid sequence having at least 90% (e.g., at least 90%, 91%, 92%, 93%, 94%, 95%, 96%, 97%, 98%, 99%, or more) sequence identity to SEQ ID NO: 5. In certain embodiments, the Cusativin enzyme is encoded by a nucleic acid sequence having at least 95% (e.g., at least 95%, 96%, 97%, 98%, 99%, or more) sequence identity to SEQ ID NO: 5. In certain embodiments, the Cusativin enzyme is encoded by a nucleic acid sequence having at least 99% (e.g., at least 99%, or more) sequence identity to SEQ ID NO: 5. In certain embodiments, the Cusativin enzyme is encoded by a nucleic acid sequence of SEQ ID NO: 5.

(SEQ ID NO: 5)
ATGGAAAAGACCAAAAGTGTCGATGTGGTGTTTTTTGTATTTGTCCTGAC
TATCCTGTTCCCGATCGTCAAGAGTCAGACATTTGACGATTTCTGGTTTG
TTCAACAATGGCCTCCGGCGGTTTGTACGTTACAGTCTGGCCGCTGTGTT
GGTCGTGGAACTCGCTCATTTACTATTCATGGCCTGTGGCCACAGAAAGG
CGGCCGTAGTGTTACGAACTGTACTGGTAACCAGTTTGATTTTACGAAAA
TTGCACACCTGGAAAACGATTTGAACGTTGTATGGCCCAATGTGGTCACG
GGCAATAACAAATTCTTTTGGGGGCATGAGTGGAATAAGCACGGCATCTG
TTCAGAATCCAAATTTGATGAAGCCAAATATTTCCAGACCGCGATTAACA
TGCGCCATGGCATCGATCTGTTGAGCGTCCTCCGTACCGGCGGTGTTGGT
CCAAATGGCGCTAGCAAAGCCAAACAACGCGTGGAAACCGCCATCTCCTC
CCACTTTGGTAAGGATCCGATTCTGCGGTGCAAAAAGGCGTCGAACGGCC
AAGTGCTGCTGACGGAGATTGTGATGTGCTTTGATGACGACGGCGTCACC
CTGATCAATTGTAATAAGGCGCGCTCCAATTGCGCAGGCTCATTTATTTT
T

### EXAMPLES

The following examples are put forth to provide those of ordinary skill in the art with a description of how the methods described herein may be used, made, and evaluated, and are intended to be purely exemplary of the disclosure and are not intended to limit the scope of what the inventors regard as their invention.

### General Methods

### Buffer Preparation

### 200 mM Ammonium Acetate Buffer, pH 6.8 (not Adjusted)

The 200 mM ammonium acetate buffer was prepared as follows. A stock solution of 5 M ammonium acetate was diluted to 200 mM with 18.2 MΩ water. The buffer was stored at 4° C.

### 200 mM Ammonium Acetate Buffer, pH 8.0

The 200 mM ammonium acetate buffer, pH 8.0, was prepared as follows. A stock solution of 5 M ammonium acetate was diluted to 200 mM with 18.2 MΩ water, and pH was adjusted to 8.0 with ammonium hydroxide. The buffer was stored at 4° C.

### 200 mM Ammonium Acetate Buffer, pH 9.0

The 200 mM ammonium acetate buffer, pH 9.0, was prepared as follows. A stock solution of 5 M ammonium acetate was diluted to 200 mM with 18.2 MΩ water, and pH was adjusted to 9.0 with ammonium hydroxide. The buffer was stored at 4° C.

### Substrate RNA Preparation

### Substrate RNA 35-Mer Preparation

A customized RNA sequence of a 35-mer RNA polynucleotide having the sequence of GCAUCAGAAAAUACACCCCGUAGGGGCUUUUGAGA (SEQ ID NO: 39) was synthesized. The final yield was 217 nmol. The RNA was resuspended in 1000 μL to prepare a concentration of 217 pmol/μL. 100 μL was diluted with 900 μL water to get ˜20 pmol/μL. 5 μL or 100 pmol substrate RNA used for RNA digestion.

Substrate RNA Preparation: Hs.Cas9.HPRT1.1.AA sgRNA

A catalogue item for sgRNA Hs.Cas9.HPRT1.2.AA having the sequence of mG*mA*mU*GAUCUCUCAACUUUAACGUUUUAGAGCUAGAAAUAGCAAGUUAA AAUAAGGCUAGUCCGUUAUCAACUUGAAAAAGUGGCACCGAGUCGGUGCmU*mU*mU*U (SEQ ID NO: 10) was procured (Average MW: 32,275 Da). mG*, mA*, and mU* represent 2′-O-methylguanosine, 2′-O-methyladenosine, and 2′-O-methyluridine with phosphorothioate linkers. One vial containing 100.0 nmol sgRNA sample was rehydrated with 1 mL of freshly collected 18.2 MΩ water to achieve 100 pmol/μL solution. The RNA solution was aliquoted and frozen at −20° C. for long-term storage. When needed, the RNA aliquots were thawed at either ambient temperature or 4° C., vortexed briefly, and spun on a tabletop microfuge to achieve a homogeneous suspension.

### RNA Digestion Protocol

In general, 100±1 pmol of 35-mer or sgRNA from stock (100 pmol/μL, 1 μL, ˜3 μg) was added to 10 μL 200 mM ammonium acetate (with or without adjusted pH specific to each enzyme used) for RNA digestion. The RNA solution was exposed to 90° C. for 2 min and cooled by keeping it on ice or cold rack that maintains 4° C. The requisite units (50 U) of enzyme were added in 10 μL, depending on the enzyme and RNA substrate, to obtain final volume of 20 μL. The samples were gently vortexed and centrifuged in a microfuge and incubated in a Thermomixer for 1 hour at 30° C., followed by exposure to 70° C. for 15 min to inactivate and terminate the enzyme-mediated digestion. The reaction mixture was then transferred to polypropylene LC-MS vials for oligonucleotide mapping analysis.

### Liquid Chromatography-Mass Spectrometry (LC-MS)

### Mobile Phase Preparation for IP-RP-LC

Mobile phase A was. 1% N,N-diisopropylethylamine (DIPEA) and 1% 1,1,1,3,3,3-hexafluoroisopropanol (HFIP) in 18.2 MΩ water. Mobile phase A was prepared by adding 2.0±0.05 mL of DIPEA and 20±0.1 mL of HFIP to 1978±0.5 mL of 18.2 MΩ water and sonicating for 15-20 min to prepare a uniform solution. Mobile phase B was 0.0375% DIPEA and 0.075% HFIP in 55:10:35 Acetonitrile:Methanol: 18.2 M62 water. Mobile phase B was prepared by adding 0.375±0.010 mL of DIPEA, 0.75±0.010 mL of HFIP, 100±0.1 mL of methanol, and 550±0.1 mL of acetonitrile to 349±0.1 mL of 18.2 MΩ water and sonicating for 15-20 min. Sample wash, seal wash, and purge solvents were prepared by adding 400±1 mL of acetonitrile in a 1 L graduated cylinder to 1600±1 mL of 18.2 MΩ water in a 2 L reservoir bottle and sonicated for 15 min. Sample wash, seal wash, and purge solvent lines were inserted for purging. Solutions were stored at room temperature until needed for use. Cleaning solvent containing 25% Isopropanol, 25% Methanol, 25% Acetonitrile, and 25% 18.2 MΩ water was prepared as follows. 250±1 mL of isopropanol was added in a 1 L Class A graduated cylinder and transferred to a 1 L reservoir bottle. 250±1 mL of methanol, 250±1 mL of acetonitrile, and 250 mL of 18.2 MΩ water were added to the 1 L reservoir bottle and mixed well. Solutions were stored at room temperature until needed for use. Lock mass solution containing leucine enkephalin lyophilized peptide was prepared as follows. A sealed ampule of Leucine Enkephalin was opened into a bottle containing 375 mL of 50/50 v/v ACN/water and 750 μL formic acid, recapped, shaken well, and sonicated for 5 min. The devices and settings shown in Table 1, below, were used for LC-MS experiments, including IP-RP-LC-UV-MS settings.

TABLE 1
IP-RP-LC-UV-MS Settings

| System: | ACQUITY UPLC ® H-Class plus Bio System [Consisting of a | |
|---|---|---|
| | BSM with 50 μL Mixer (700012635) SN: D20BBS000N, TUV | |
| | Detector (Flow cell: Titanium, 5 mm, 1500 nL), SN: | |
| | K10TUV352A, FTN-SM with HPS 15 μL MP35N P-4 Needle | |
| | p/n: 700005421, SN: L19FBP580M, CH-30A heater with an | |
| | Active Preheater 18.5″ p/n: 205002234 (279004352) HPS and | |
| | post-column tubing to TUV: 0.0025″ ID × 22.5″ LG MP35N | |
| | Tube p/n: 430001749] | |
| Data Acquisition | Waters_Connect (version: 4.1.0.17), Unifi (version: | |
| and Analysis: | 3.8.0.23) | |
| Column: | ACQUITY Premier Oligonucleotide BEH C18 Column, 300 Å, | |
| | 1.7 μm, 2.1 × 50 mm, p/n: 186010541 or equivalent (SN: | |
| | 01572302620A03) | |
| Temperature: | Column temperature: 70° C. | |
| Seal Wash: | 20% HPLC grade acetonitrile/80% 18.2 MΩ water (v/v) (Seal | |
| | Wash interval set to 0.5 min) | |
| Sample Manager | 18.2 MΩ water | |
| Washes: | | |
| Mobile Phase A1: | 0.1% N,N-diisopropylethylamine (DIPEA) as the IP reagent and | |
| | 1% 1,1,1,3,3,3-hexafluoroisopropanol (HFIP) in 18.2 MΩ water | |
| Mobile Phase B1: | 0.0375% DIPEA and 0.075% HFIP in 55:10:35 Acetonitrile: | |
| | Methanol:18.2 MΩ water | |
| Mobile Phase A2: | 100% Acetonitrile | |
| Mobile Phase B2: | 100% Acetonitrile | |
| Active Preheater: | Enabled in ACQUITY-FTN method | |
| Test Flow Rate | 0.4 | mL/min |
| Sample Temperature: | 8° | C. |
| Sample Injection: | Refer to example Sample Set | |
| Syringe Draw Rate: | 30 | μL/min |
| Needle Placement: | 1.0 | mm |
| Air Gaps: | Not checked (none) | |
| Data Channels: | System Pressure and TUV | |
| UV Wavelength: | 254 | nm |
| TUV Sampling Rate: | 5 | Hz |

| | |
|---|---|
| Filter Time Constant: | none |
| Data Mode: | Absorbance |
| Autozero On Inject | Yes |
| Start: | |
| Autozero On | Maintain Baseline |
| Wavelength: | |

The LC chromatography gradient used for the LC-MS experiments is shown in Table 2, below.

TABLE 2
Chromatography gradient

| Time | Composition | Composition | Flow rate | |
|---|---|---|---|---|
| (min) | A (%) | B (%) | (mL/min) | Curve |
| 0.0 | 99.0-97.0 | 1.0-3.0 | 0.3-0.4 | Initial |
| 35.0 | 80.0 | 20.0 | 0.3-0.4 | 6 |
| 40.0 | 30.0 | 70.0 | 0.3-0.4 | 6 |
| 42.0 | 30.0 | 70.0 | 0.3-0.4 | 6 |
| 52.0 | 97.0 | 30.0 | 0.3-0.4 | 6 |

Mass spectrometry settings used in LC-MS runs are shown in Table 3, below.

TABLE 3
Mass spectrometry settings

| Mass spectrometer | Xevo G3 QTof (SN: YGA0157) | |
|---|---|---|
| | Driver:1.3.0 | |
| Source type | ESI | |
| Polarity | Negative | |
| Analyzer mode | Sensitivity | |
| Capillary Voltage | 1.5 | kV |
| Sample Cone Voltage | 40 | V |
| Source Temperature | 100° | C. |
| Desolvation Temperature | 550° | C. |
| Cone gas | 50 | L/h |
| Desolvation gas | 650 | L/h |
| Scan Mass range | 550-2000 | |
| Scan Time | 1.0 | sec |
| Collision energy: Low | 6 | V |
| High energy ramp | 10-45 | V |
| Intelligent Dat Capture- | Medium (10) | |
| Intensity Threshold | | |
| Lock correction | Automatic (30 sec intervals) | |
| Events: LC, sample | 2.0-40.0 | min |
| Lock mass: Leucine Enkephalin | 554.26202 | (M − H+)¹⁻ |

| | |
|---|---|
| Lock mass: Combine width and | 3 scans, 0.5 m/z |
| mass window | |

### Example 1: High pH Modulates Ribonuclease Activity of RNase MC1 to Produce Longer Digestion Products

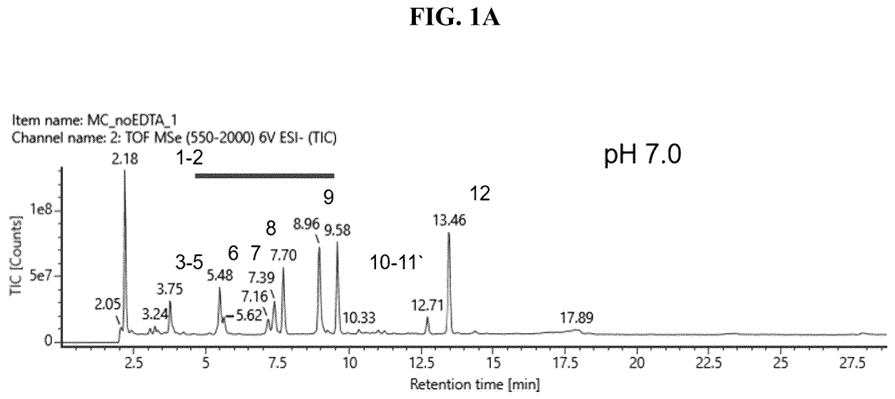

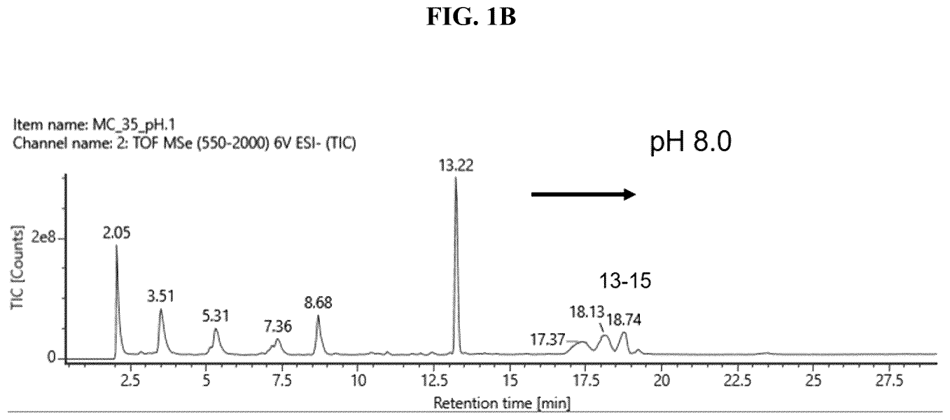

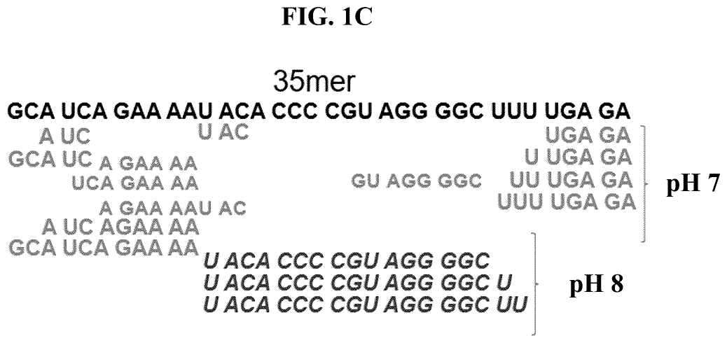

To determine the effect of pH on the RNA cleavage activity of RNase MC1, 100 pM of a 35-mer RNA polynucleotide was digested for one hour in a 20 μL reaction volume with 100 mM ammonium acetate pH 8.0 for 1 hour. 10 μL of the reaction solution was subjected to LC-MS following enzyme inactivation. Expected digestion products predicted based on the cleavage rules of MC1 (www.mdpi.com/1422-0067/23/13/7021) were generated using Waters microapp, mRNA cleaver (microapps.on-demand.waters.com/home/showmarkdown/mrna-cleaver), and mass-to-charge (m/z) values were used to score the peak IDs. RNase MC1 cleaves RNA at [A/C/U] pU and Cp [A/G] dinucleotide sites. pH IP reagents are needed in RP chromatography to ensure retention and separation of digested oligonucleotide fragments. Highly polar, negatively charged oligonucleotides form a non-covalent ion pairs with the IP reagent (positively charged amines such as triethylamine, diisopropylethylamine, etc.), and this ionic pair interacts with the hydrophobic stationary phase (C18). Longer oligonucleotides exhibit greater retention in the stationary phase, thus facilitating the size and hydrophobicity-based chromatographic separations. The total ion chromatogram of oligonucleotide digestion products for each digestion reaction is shown in FIGS. 1A and 1B. Identified peaks are numbered on the chromatogram and their IDs are shown in Table 4, below, which provides the identified digestion products and their retention time (RT) from both unadjusted and high pH conditions (peak numbers correspond to peaks numbered in FIG. 1A and FIG. 1B).

TABLE 4
Identified digestion products and
their retention times

| Peak | Digest Sequence | Retention Time (min) |
|---|---|---|
| 1 | GCAUC | 2.18 |
| 2 | UGAGA-OH | 2.18 |
| 3 | UAC | 3.78 |
| 4 | AUC | 3.78 |
| 5 | UUUGAGA | 3.78 |
| 7 | AGAAAA | 5.38 |
| 6 | UUUUGAGA | 5.66 |
| 8 | GUAGGGGC | 7.36 |
| 9 | UCAGAAAA | 8.96 |
| 10 | AGAAAAUAC | 11.24 |
| 11 | AUCAGAAAA | 11.24 |
| 12 | GCAUCAGAAAA | 13.46 |
| | (SEQ ID NO: 6) | |
| 13 | UACACCCCGUAGGGGC | 18.09 |
| | (SEQ ID NO: 7) | |
| 14 | UACACCCCGUAGGGGCU | 18.52 |
| | (SEQ ID NO: 8) | |
| 15 | UACACCCCGUAGGGGCUU | 19.1 |
| | (SEQ ID NO: 9) | |

Overlay of observed digestion products mapped onto the RNA sequence, as illustrated in FIG. 1C, shows longer digestion products observed with high pH digestion with RNase MC1. Indeed, shorter products (≤11-mers) were observed under unadjusted pH conditions (pH 7), while their abundance was reduced with high pH (pH 8) digestion condition. Further, longer digestion products (>11-mers) were observed when pH was increased in the digestion mix. The substrate amount, enzyme units, and ionic strength of the buffer were kept constant in both sets of experiments.

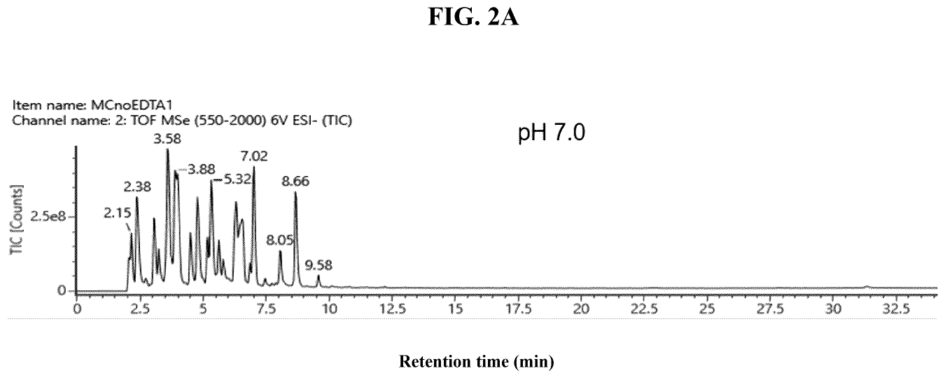

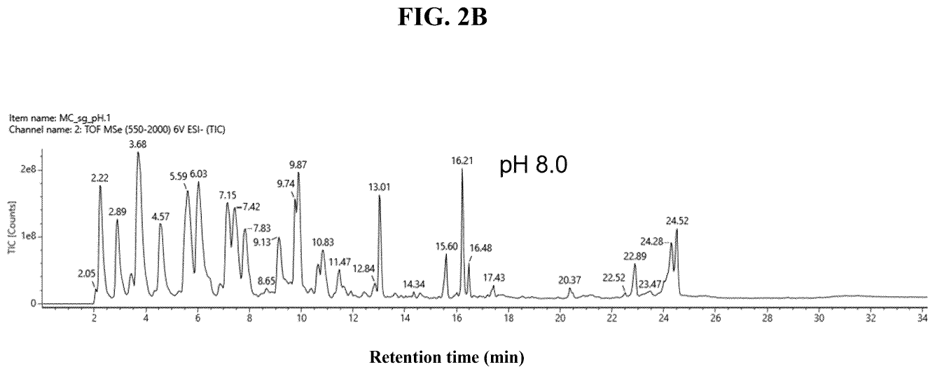

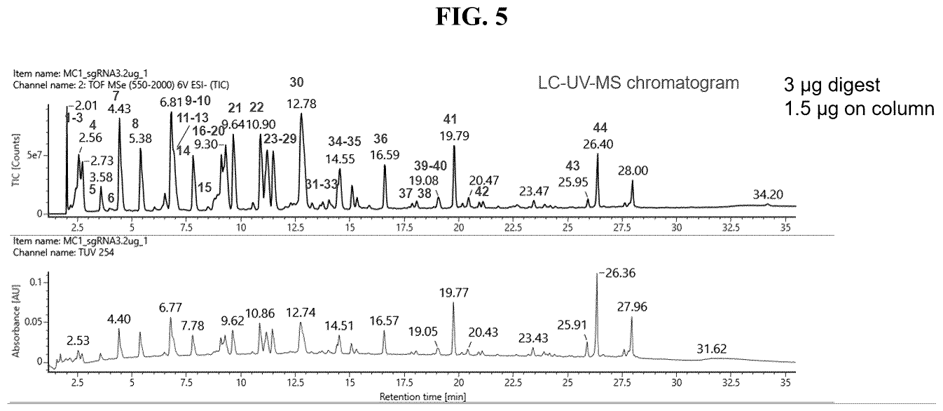

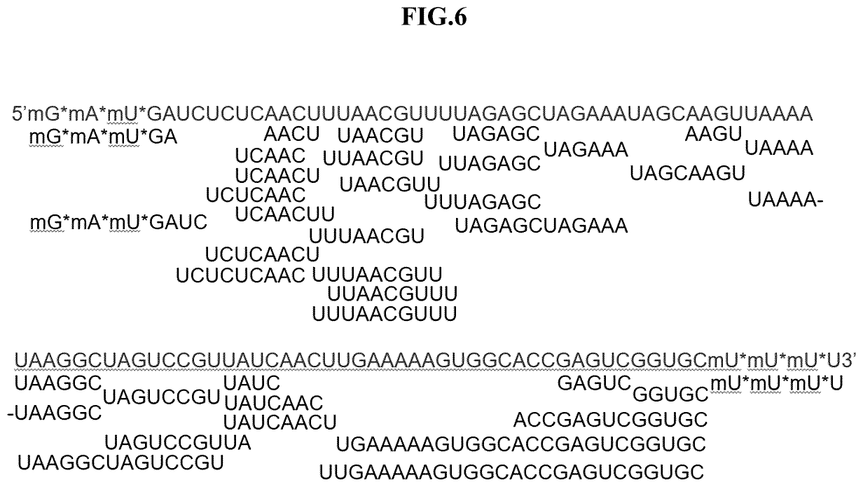

In a set of related experiments, RNase digestion of a single guide RNA (sgRNA) targeting Hypoxanthine Phosphoribosyltransferase (HPRT) was tested under unadjusted (pH 7) and high pH (pH 8) conditions. 100 pmol (˜3 μg) of HPRT sgRNA was digested the presence of 50 U of MC1 enzyme and 100 mM ammonium acetate (pH 7.0, FIG. 2A) or pH adjusted to 8.0 (FIG. 2B and FIG. 5) for 1 hour and subjected to IP-RP-LC-MS or LC-UV-MS. Total ion chromatogram of sgRNA under unadjusted pH conditions showed the presence of shorter digestion products with retention time <10 min (FIG. 2A). Longer digestion products (13.22 min-24.52 min) were detected when digestion was carried out under high pH conditions (FIG. 2B and FIG. 5A, top panel). The peak IDs were generated based on in silico prediction and manual identification in a workflow identical to that of the 35-mer. The mass error for screening was pegged at <20 ppm. The UV trace of the oligonucleotide digestion products is shown in FIG. 5 (bottom panel). The generated digestion products exhibited an abundant UV signal that could be suitable for quality control studies where MS may not be employed. Overall, 44 digestion products of varying lengths and retention times were scored and annotated on the chromatogram, as shown in Table 5, below. This table denotes the sequence of oligonucleotide digestion product, location in the sequence, monoisotopic neutral mass, a representative m/z value specific to each oligomer and the observed retention time. An overlay of the identified digestion products mapped onto the HPRT sgRNA sequence is shown in FIG. 6.

TABLE 5
RNase MC1-produced oligonucleotide digestion fragments of HPRT sgRNA at
pH 8.0

| | | | Monoisotopic | | Retention time |
|---|---|---|---|---|---|
| Peak | Item Name | Location | mass | m/z | (min) |
| 1 | UAUCcP | U64:C67 | 1246.1444 | 622.0649 | 2.15 |
| 2 | AAGUcP | A41:U44 | 1309.1778 | 653.5816 | 2.18, 2.35 |
| 3 | AACUcP | A12:U15 | 1269.1716 | 633.5785 | 2.18 |
| 4 | mU*mU*mU*U | 197:U100 | 1252.1238 | 625.0546 | 2.56-2.73 |
| 5 | GGUGCCP | G92:C96 | 1646.2089 | 822.0972 | 3.58 |
| 6 | GAGUCcP | G87:C91 | 1630.2140 | 814.0997 | 3.95 |
| 7 | UCAACCP | U10:C14 | 1574.2129 | 786.0992 | 4.43 |
| 8 | UAAAACP | U45:A49 | 1621.2354 | 810.1104 | 5.38 |
| 9 | UAGAGCcP | U25:C30 | 1959.2665 | 978.6260 | 6.81 |
| 10 | UAAGGCCP | U50:C55 | 1959.2665 | 978.6260 | |
| 11 | UAACGUcP | U17:U22 | 1920.2444 | 959.1149 | 7 |
| 12 | UCAACUcP | U10:U15 | 1880.2382 | 939.1118 | |
| 13 | UUAACGUcP | U16:U22 | 2226.2697 | 1112.1276 | 7.43 |
| 14 | UAGAAAcP | U31:A36 | 1967.2828 | 982.6341 | 7.8 |
| 15 | mG*mA*mU*GAcP | B1:A5 | 1744.2036 | 871.0945 | 8.79-8.99 |
| 16 | UUAGAGCcP | U24:C30 | 2265.2918 | 1131.6386 | 9.1 |
| 17 | UUAACGUcP | U16:U22 | 2226.2697 | 1112.1276 | 9.23 |
| 18 | UAACGUUCP | U17:U23 | 2226.2697 | 1112.1276 | |
| 19 | UCUCAACCP | U8:C14 | 2185.2795 | 1091.6325 | 9.3 |
| 20 | UCAACUUCP | U10:U16 | 2186.2635 | 1092.1245 | |
| 21 | UAUCAACCP | U64:C70 | 2209.2907 | 1103.6381 | 9.64 |
| 22 | UAGUCCGUcP | U56:U63 | 2547.3059 | 1272.6457 | 10.86 |
| 23 | UUUAGAGCCP | U23:C30 | 2571.3171 | 1284.6513 | 11.11 |
| 24 | UUUAACGUcP | U15:U22 | 2532.2950 | 1265.1402 | 11.21 |
| 25 | UUAACGUUCP | U16:U23 | 2532.2950 | 1265.1402 | |
| 26 | UAACGUUUcP | U17:U24 | 2532.2950 | 1265.1402 | |
| 27 | UCUCAACUcP | U8:U15 | 2491.3048 | 1244.6451 | 11.24 |
| 28 | UAGCAAGUcP | U37:U44 | 2594.3443 | 1296.1649 | 11.48 |
| 29 | UAUCAACUcP | U64:U71 | 2515.3160 | 1256.6507 | 11.59 |
| 30 | mG*mA*mU*GAUCcP | B1:C7 | 2355.2701 | 1176.6278 | 12.78 |
| 31 | UCUCUCAACCP | U6:C14 | 2796.3461 | 1397.1658 | 13.05 |
| 32 | UUUAACGUUCP | U15:U23 | 2838.3203 | 1418.1529 | |
| 33 | UUAACGUUUcP | U16:U24 | 2838.3203 | 1418.1529 | |
| 34 | UUUAACGUUUCP | U15:U24 | 3144.3456 | 1571.1655 | 14.44 |
| | (SEQ ID NO: 11) | | | | |
| 35 | UAGUCCGUUAcP | U56:A65 | 3182.3837 | 1590.1846 | 14.55 |
| | (SEQ ID NO: 12) | | | | |
| 36 | UAAAAUAAGGCCP | U45:C55 | 3581.5019 | 1789.7437 | 16.59 |
| | (SEQ ID NO: 13) | | | | |
| 37 | UAGAGCUAGAAAcP | U25:A36 | 3926.5493 | 1962.2674 | 17.9 |
| | (SEQ ID NO: 14) | | | | |
| 38 | ACCGAGUCGGUGCcP | A84:C96 | 4215.5580 | 1404.1787 | 18.09 |
| | (SEQ ID NO: 15) | | | | |
| 39 | UAAGGCUAGUCCGUcP | U50:U63 | 4506.5724 | 1501.1835 | 19.08 |
| | (SEQ ID NO: 16) | | | | |
| 40 | UAGCAAGUUAAAAcP | U37:A49 | 4216.5797 | 1404.5193 | 19.14 |
| | (SEQ ID NO: 17) | | | | |
| 41 | UAGAAAUAGCAAGUCP | U31:U44 | 4561.6271 | 1519.5351 | 19.79 |
| | (SEQ ID NO: 18) | | | | |
| 42 | UAAGGCUAGUCCGUUAcP | U50:A65 | 5141.6502 | 1712.8761 | 20.95 |
| | (SEQ ID NO: 19) | | | | |
| 43 | UGAAAAAGUGGCACCGA | U72:C96 | 8158.1022 | 1630.6132 | 25.95 |
| | GUCGGUGCCP | | | | |
| | (SEQ ID NO: 20) | | | | |
| 44 | UUGAAAAAGUGGCACCG | U71:C96 | 8464.1275 | 1691.8182 | 26.4 |
| | AGUCGGUGCCP | | | | |
| | (SEQ ID NO: 21) | | | | |

“cP” represents cyclic phosphate.

These results indicate that this type of partial cleavage which generates overlapping digestion products eliminates the need for multiple digests of same RNA with more than one enzyme. Ribonucleases such as RNase TI traditionally generate discreet number of digestion products with no overlapping features, thereby losing the global sequence context. Partial digestion of RNA by enzymes such as MC1 facilitates generation of digestion products with overlapping features, thereby providing sequence context and confident data interpretation, as a given sequence location is represented more than once through multiple products in the same digestion reaction.

### Example 2: Ethylenediaminetetraacetic Acid (EDTA) Modulates Ribonuclease Activity of Rnase MC1 to Produce Longer Digestion Products

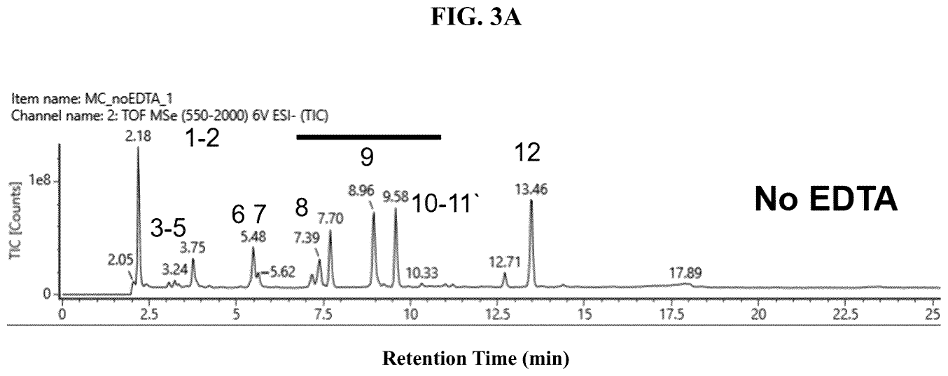

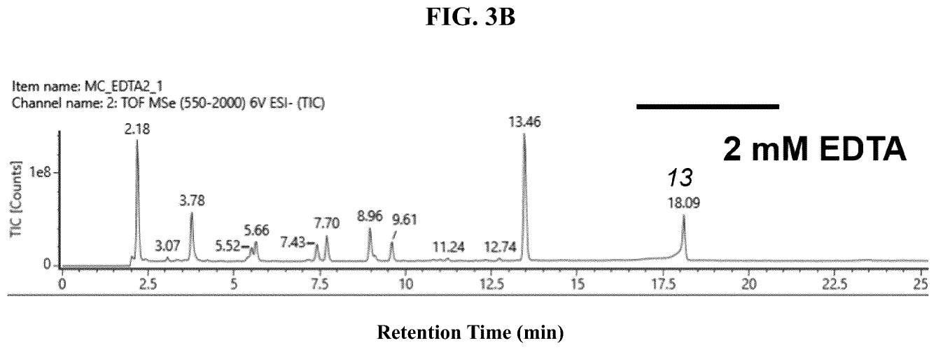

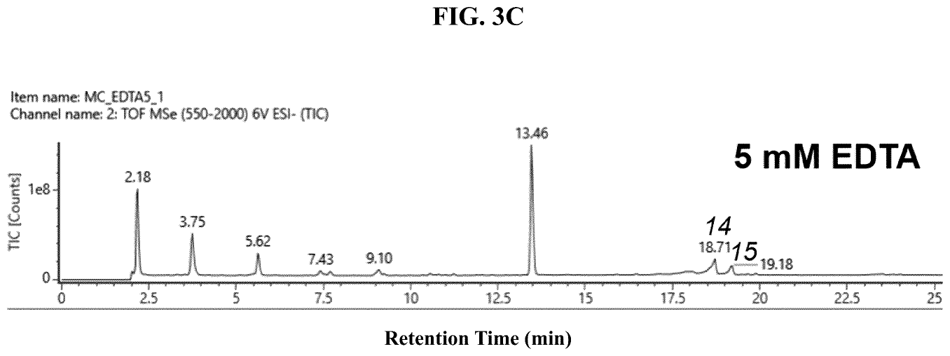

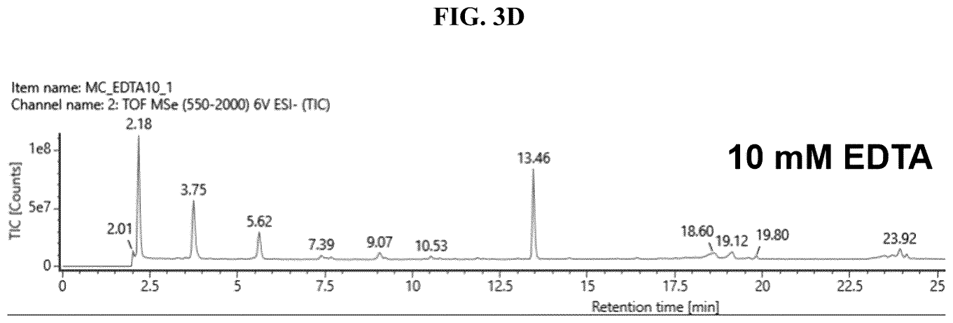

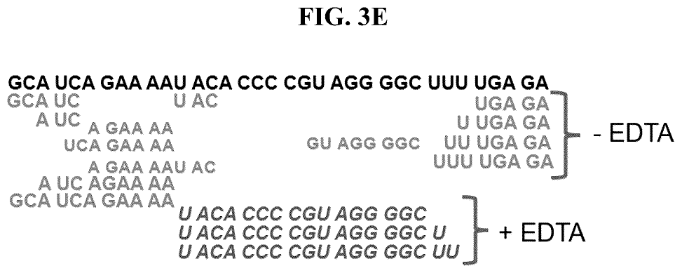

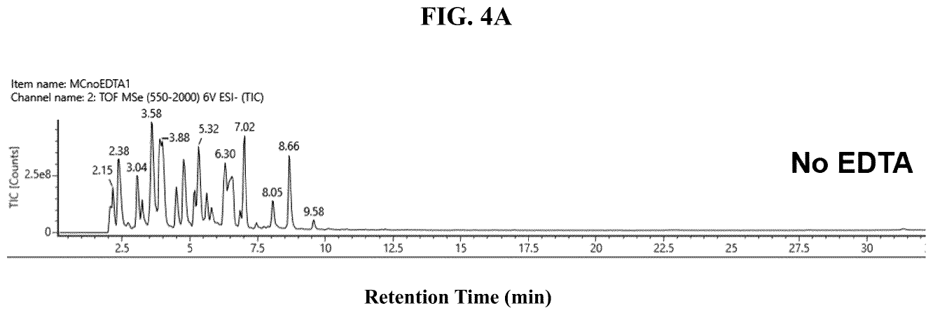

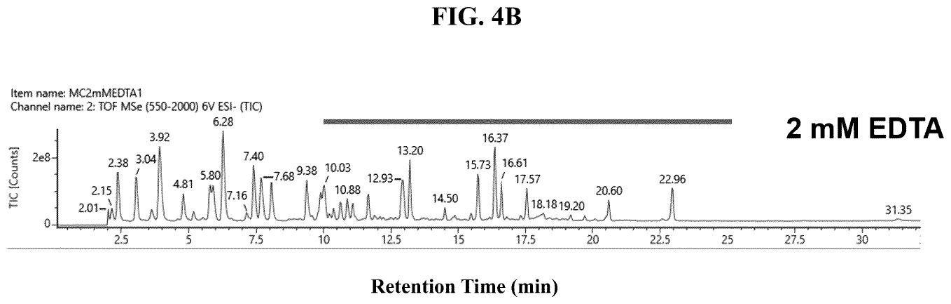

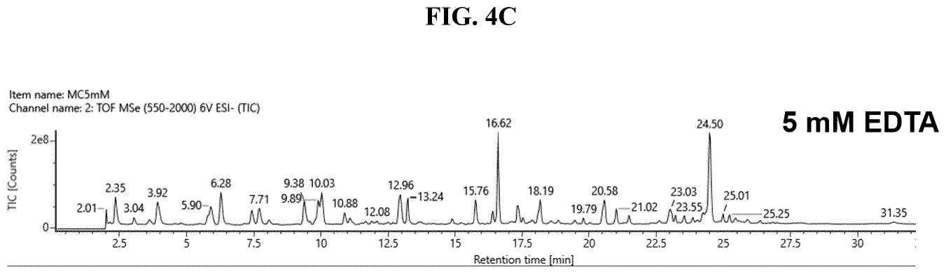

To determine the effect of inclusion of particular additives on the RNA cleavage activity of Rnase MC1, a 35-mer RNA polynucleotide was digested in the presence of 100 mM ammonium acetate (pH 7.0) without EDTA or with varying concentrations of EDTA (2 mM, 5 mM, or 10 mM) and subjected to IP-RP-LC-MS. Total ion chromatograms of 35-mer RNA digests performed without and with EDTA are shown in FIGS. 3A-3D. (FIG. 3A) No EDTA, (FIG. 3B) 2 mM, (FIG. 3C) 5 mM, and (FIG. 3D) 10 mM EDTA. A drop in intensities of peaks 8-11 (<10 min retention time) as compared to the EDTA-free samples was observed as EDTA concentration increased. This was accompanied by the appearance of peaks 13-15 at elevated EDTA conditions. Interestingly, the peak at 13.46 min was unchanged despite EDTA presence indicating high susceptibility of this bond for MC1 cleavage (FIGS. 4B-4C). Table 6, below, shows a list of all oligonucleotide digestion products detected from the RNA digests along with their retention time, and FIG. 4E provides an overlay of predominately detected oligonucleotide digestion products mapped to the nucleotide sequence of the 35-mer RNA polynucleotide.

TABLE 6
Rnase MC1-generated oligonucleotide digestion products of a
35-mer RNA polynucleotide in the presence and absence of EDTA

| Peak | Sequence | Retention Time (min) |
|---|---|---|
| 1 | GCAUC | 2.18 |
| 2 | UGAGA-OH | 2.18 |
| 3 | UAC | 3.78 |
| 4 | AUC | 3.78 |
| 5 | UUUGAGA | 3.78 |
| 6 | UUUUGAGA | 5.66 |
| 7 | AGAAAA | 5.38 |
| 8 | GUAGGGGC | 7.36 |
| 9 | UCAGAAAA | 8.96 |
| 10 | AGAAAAUAC | 11.24 |
| 11 | AUCAGAAAA | 11.24 |
| 12 | GCAUCAGAAAA | 13.46 |
| | (SEQ ID NO: 22) | |
| 13 | UACACCCCGUAGGGGC | 18.09 |
| | (SEQ ID NO: 23) | |
| 14 | UACACCCCGUAGGGGCU | 18.52 |
| | (SEQ ID NO: 24) | |
| 15 | UACACCCCGUAGGGGCUU | 19.1 |
| | (SEQ ID NO: 25) | |

In a set of related experiments, Rnase MC1-mediated digestion of HPRT sgRNA was tested in the absence of EDTA or with varying concentrations of EDTA. HPRT sgRNA was digested in the presence of 100 mM ammonium acetate (pH 7.0) without EDTA or with 2 mM or 5 mM EDTA and subjected to IP-RP-LC-MS. Total ion chromatogram of sgRNA showed a change in oligonucleotide peak profiles between EDTA-free condition (FIG. 4A) and EDTA-containing conditions (FIGS. 4B-4C). In the absence of EDTA, smaller digestion products with shorter retention times (<10 min) were observed (FIG. 4A). As the EDTA concentration increased, peaks beyond 10 min retention time were observed, representing longer digestion products (FIGS. 4B-4C). Table 7, below, provides a list of representative oligonucleotide digestion products that matched with the expected cleavages of MC1 on sgRNA sequence.

TABLE 7
Representative Rnase MC1-produced HPRT sgRNA
fragments in the absence or presence of EDTA

| | | |
|---|---|---|
| Peak | Sequence | Retention Time (min) |

No EDTA and +EDTA digests

| | | |
|---|---|---|
| 1 | UCAAC | 2.38 |
| 2 | UAAAA | 3.04 |
| 3 | UAACGU | 3.99 |
| 4 | UAGAAA | 4.77 |
| 5 | UUAACGU | 5.8 |
| 6 | UUAGAGC | 5.8 |
| 7 | UAUCAAC | 6.3 |
| 8 | UAGCAAGU | 8.05 |

+EDTA digests

| | | |
|---|---|---|
| 10 | mG*mA*mU*GAUCUC | 12.93 |
| 11 | UAAAAUAAGGC | 13.2 |
| | (SEQ ID NO: 26) | |

mG*, mA*, mU* represent 2′-O-methylguanosine, 2′-O-methyladenosine, and 2′-O-methyluridine with phosphorothioate linkers.

### Example 3: Combination of High pH and EDTA Further Enhances Rnase MC1 Enzyme Activity to Generate Longer Digestion Products

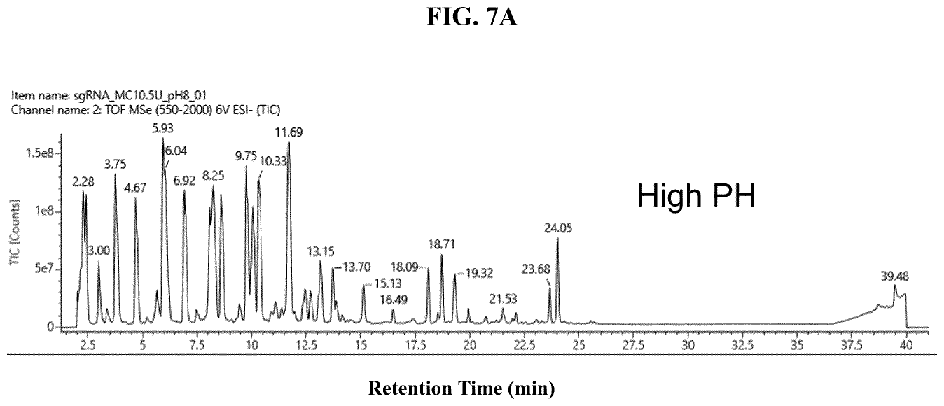

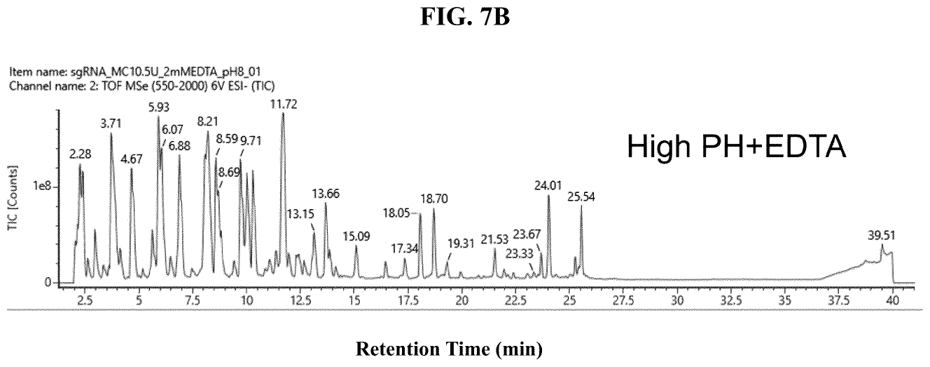

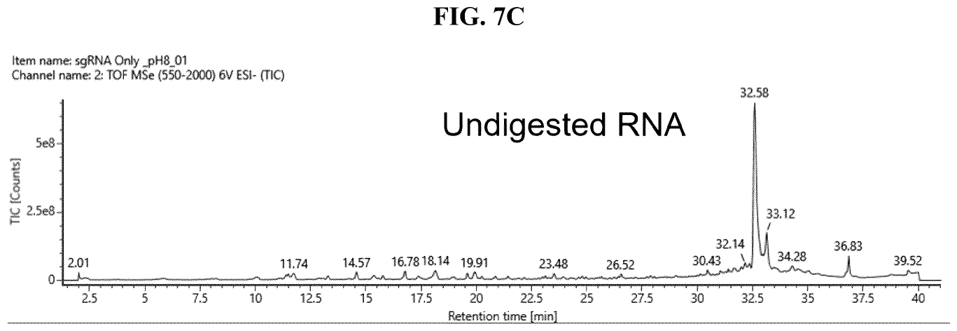

To determine the combined effect of high pH and presence of EDTA on RNA cleavage of Rnase MC1, HPRT sgRNA was digested with MC1 at pH 8.0 in the absence (FIG. 7A) or presence of 2 mM EDTA (FIG. 7B) at pH 8.0 and subjected to IP-RP-LC-MS. Combination of high pH and EDTA resulted in the appearance of longer digestion products with elution times beyond 24 min (FIG. 7B). However, the peak pattern of digestion products observed at elution times below 24 min remained unchanged either in the absence or presence of EDTA (FIGS. 7A-7B), suggesting that site-selectivity of cleavages was unchanged, but the activity was tunable to generate longer digestion products. Elution profile of undigested sgRNA was used as a reference (FIG. 7C).

### Example 4: Thermal Modulation of Rnase MC1 Enzymatic Activity

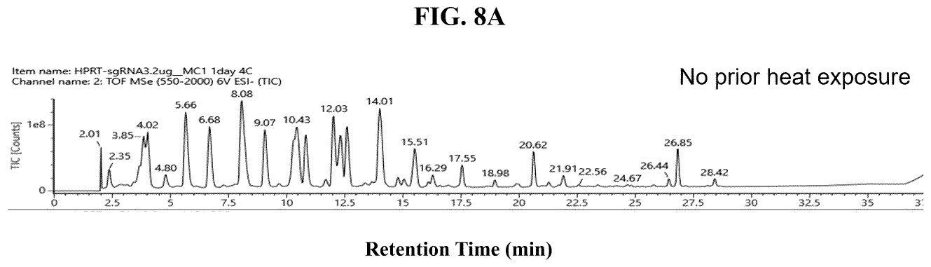

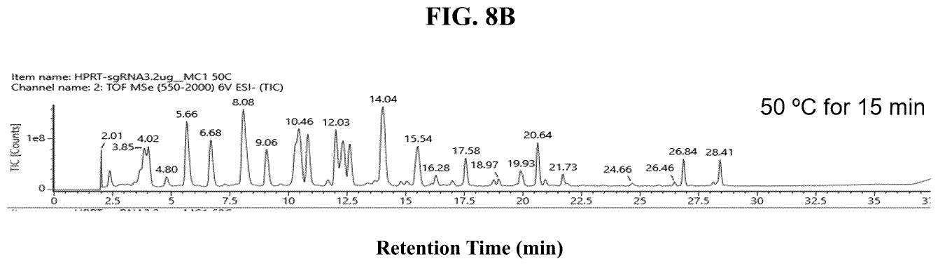

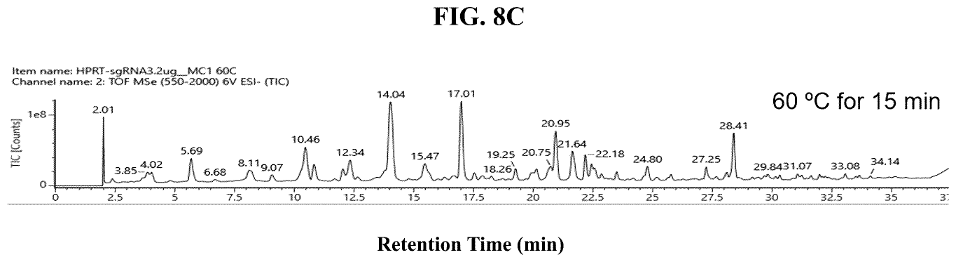

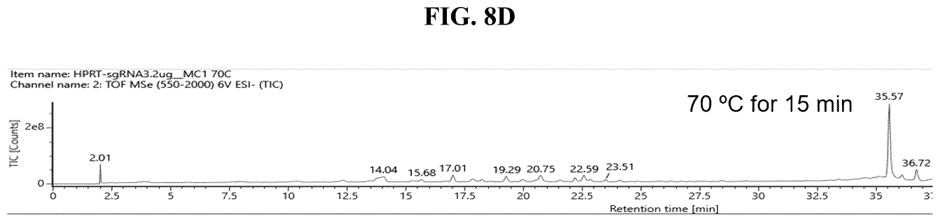

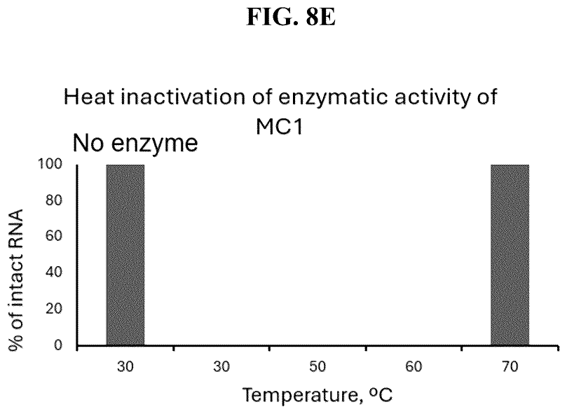

To determine the effect of reaction temperature on RNA digestion by Rnase MC1 (RapiZymes MC1), an enzyme solution containing HPRT sgRNA suspended in 200 mM ammonium acetate at pH of 8 was subjected to ambient temperature (FIG. 8A), 50° C. (FIG. 8B), 60° C. (FIG. 8C), or 70° C. (FIG. 8D) for 15 min followed by digestion of sgRNA at 30° C. for 1 hour. Prior exposure of MC1 to 50° C. for 15 min had little to no impact on the RNA digestion patterns, as indicated by an identical oligonucleotide product profile to that of no exposure (FIG. 8A vs. FIG. 8B). Exposure to 60° C. for same duration modulated the TIC profile (FIG. 8C), where some digestion products with shorter retention time (<12.5 min) exhibited decreased abundance. Moreover, an increased abundance and appearance of products with longer retention time (>12.5 min) was observed, indicating decreased activity of the enzyme. Exposure to 70° C. did not result in digestion of sgRNA, as indicated by a clear peak of intact RNA at 35.97 min (FIGS. 8D, 8E). Only negligible change in peak area was observed for MC1 enzyme exposed to 70° C. (FIG. 8E). These results indicate that Rnase MC1 exhibits temperature-dependent inactivation that becomes readily apparent at 70° C.

### Example 5: High pH Modulates Ribonuclease Activity of Cusativin to Produce Longer Digestion Products

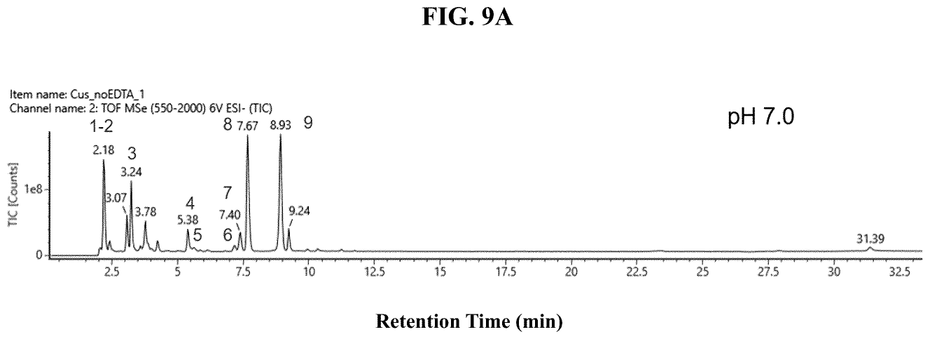

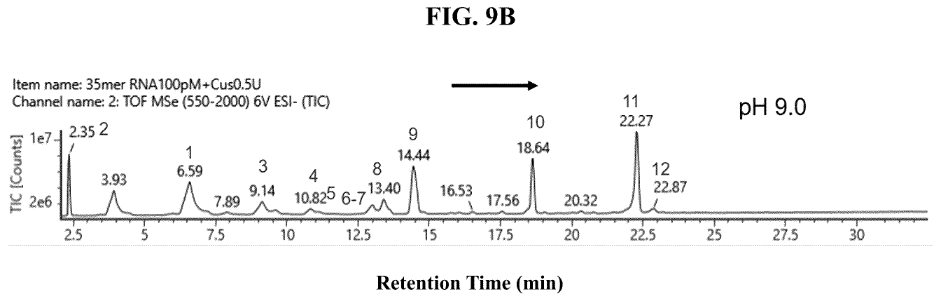

Cusativin cleaves RNA at Cp [A/G/U], [A/G] pU and Up [A/U] bonds, but the optimal conditions for cleavage specificity are unknown. To determine the effect of pH on the RNA cleavage activity of Cusativin, 100 pM of a 35-mer RNA polynucleotide was digested in a 20 μL reaction volume with 100 mM ammonium acetate (pH 7) or pH 9.0 for 1 hour. 10 μL of the reaction solution was subjected to LC-MS following enzyme inactivation. Expected digestion products predicted based on the cleavage rules of Cusativin (www.mdpi.com/1422-0067/23/13/7021) were generated using Waters microapp, mRNA cleaver (microapps.on-demand.waters.com/home/showmarkdown/mrna-cleaver). The total ion chromatogram (TIC) of oligonucleotide digestion products for each digestion reaction is shown in FIGS. 9A and 9B. Identified peaks are numbered on the chromatogram and their IDs are shown in Table 8, below, which provides the identified digestion products and their retention time (RT) from both unadjusted and high pH conditions (peak numbers correspond to peaks numbered in FIG. 9A and FIG. 9B).

TABLE 8
Cusativin-produced digestion products
of a 35-mer RNA polynucleotide

| Peak | Sequence |
|---|---|
| 1 | GCAUC |
| 2 | UGAG-OH |
| 3 | UUUGAGA-OH |
| 4 | AGAAAA |
| 5 | AGGGGCU |
| 6 | AGAAAAU |
| 7 | GUAGGGGC |
| 8 | UACACCCC |
| 9 | UCAGAAAA |
| 10 | GCAUCAGAAAA |
| | (SEQ ID NO: 27) |
| 11 | UACACCCCGUAGGGGC |
| | (SEQ ID NO: 28) |
| 12 | UACACCCCGUAGGGGCU |
| | (SEQ ID NO: 29) |

Digestion at pH 7.0 led to generation of 9 different digestion products, with an 8-mer being the longest. However, digestion at pH 9.0 led to generation of 3 additional digestion products, with a 17-mer being the longest. Although the initial gradient conditions (initial 97% A for FIG. 9A, 99% A for FIG. 9B) and flow rates (0.4 mL/min for FIG. 9A and 0.3 mL/min for FIG. 9B) were different for these two experiments, the oligomers could easily be distinguished and identified by monitoring the mass spectra.

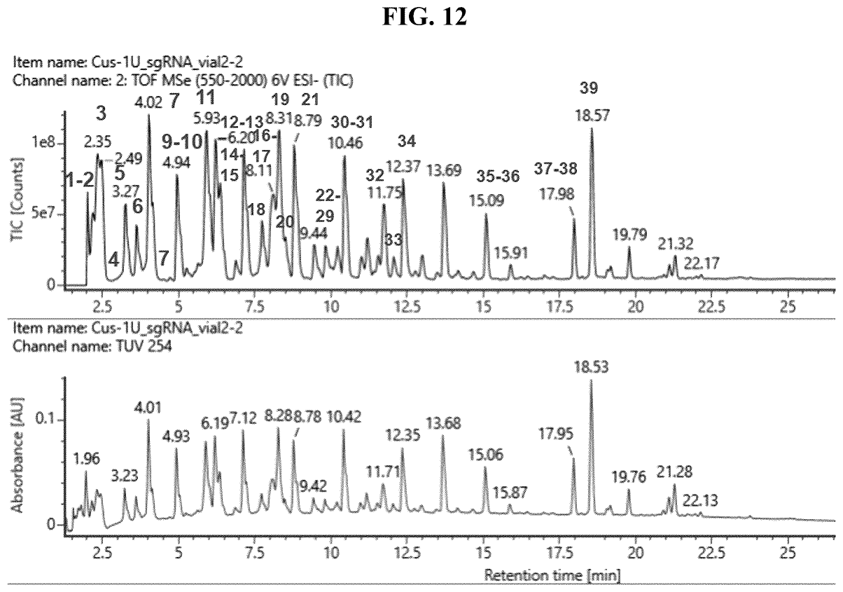

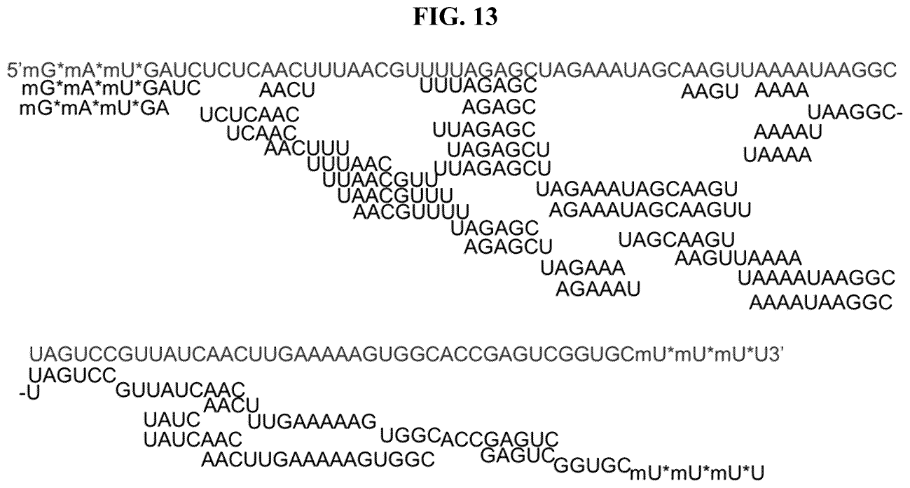

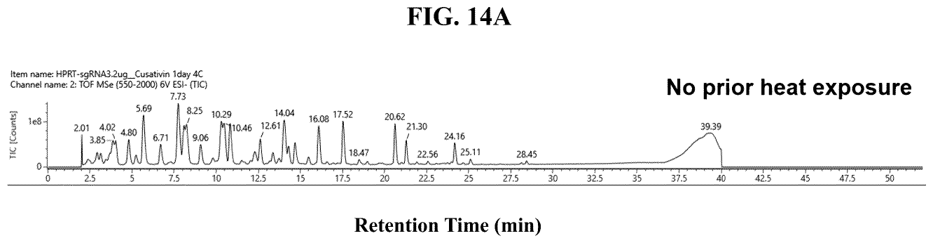

In a related set of experiments, 100 pmol (˜3 μg) of HPRT sgRNA was digested with Cusativin at pH 9 for 1 hour and subjected to IP-RP-LC-MS or LC-UV-MS. Total ion chromatogram of sgRNA under elevated pH conditions show that long digestion products (>10 min) were detected (FIG. 12, top panel). The peak IDs were generated based on in silico prediction and manual identification in a workflow identical to that of the sgRNA. The UV trace of the oligonucleotide digestion products is shown in the bottom panel of FIG. 12. Table 9, below, shows a list of identified oligonucleotide digestion products from the above experiments. This table denotes the sequence of oligonucleotide digestion product, location in the sequence, monoisotopic neutral mass, a representative m/z value specific to each oligomer and the observed retention time. An overlay of the identified digestion products mapped onto the HPRT sgRNA sequence is shown in FIG. 13.

TABLE 9
Cusativin-produced oligonucleotide digestion fragments of HPRT sgRNA at
pH 9.0

| | | | Monoisotopic | | Retention |
|---|---|---|---|---|---|
| Peak | Sequence | Location | mass | m/z | time (min) |
| 1 | AACUcP | A12:U15 | 1269.1716 | 1268.1644 | 2.05 |
| 2 | AAGUcP | A41:U44 | 1309.1778 | 1308.1705 | 2.15 |
| 3 | mU*mU*mU*U | 197:U100 | 1252.1238 | 1251.1166 | 2.35 & 2.49 |
| 4 | AAAACP | A46:A49 | 1316.2101 | 1315.2028 | 2.93 |
| 5 | GGUGCcP | G92:C96 | 1646.2089 | 1645.2020 | 3.27 |
| 6 | GAGUCcP | G87:C91 | 1630.2140 | 1629.2070 | 3.61 |
| 7 | UCAACCP | U10:C14 | 1574.2129 | 1573.2056 | 4.02 |
| 8 | AGAGCcP | A26:C30 | 1653.2412 | 1652.2339 | 4.23 |
| 9 | UAAAAcP | U45:A49 | 1622.2354 | 1621.2281 | 4.94 |
| 10 | AAAAUcP | A46:U50 | 1622.2354 | 1621.2281 | |
| 11 | UAGUCCCP | U56:C61 | 1896.2331 | 947.1093 | 5.93 |
| 12 | UAGAGCCP | U25:C30 | 1959.2665 | 978.6260 | 6.2 |
| 13 | AGAGCUcP | A26:U31 | 1959.2665 | 978.6260 | |
| 14 | AACUUUCP | A12:U17 | 1881.2222 | 939.6038 | 6.41 |
| 15 | UUUAACCP | U15:C20 | 1881.2222 | 939.6038 | |
| 16 | UAGAAAcP | U31:A36 | 1967.2828 | 982.6341 | 7.16 |
| 17 | AGAAAUcP | A32:U37 | 1967.2828 | 982.6341 | |
| 18 | UAGUCCp | U56:C61 | 1914.2437 | 956.1146 | 7.73 |
| 19 | mG*mA*mU*GacP | B1:A5 | 1744.2036 | 1743.1963 | 8.14 & 8.25 |
| 20 | UCUCAACCP | U8:C14 | 2185.2795 | 1091.6325 | 8.52 |
| 21 | UAUCAACCP | U64:C70 | 2209.2907 | 1103.6381 | 8.79 |
| 22 | UUAGAGCp | U24:C30 | 2283.3024 | 1140.6439 | 9.81 |
| 23 | UAGAGCUp | U25:U31 | 2283.3024 | 1140.6439 | |
| 24 | UAAGGCUP | U50:U56 | 2283.3024 | 1140.6439 | |
| 25 | UUUAGAGCCP | C11-14 | 2571.3171 | 1284.6513 | 10.15 |
| 26 | UUAGAGCUcP | C12-15 | 2571.3171 | 1284.6513 | |
| 27 | UUAACGUUcP | U16:U23 | 2532.2950 | 1265.1402 | 10.22 |
| 28 | UAACGUUUcP | U17:U24 | 2532.2950 | 1265.1402 | |
| 29 | AACGUUUUCP | A18:U25 | 2532.2950 | 1265.1402 | |
| 30 | ACCGAGUCcP | A84:C91 | 2569.3491 | 1283.6673 | 10.42 |
| 31 | UAGCAAGUcP | U37:U44 | 2594.3443 | 1296.1649 | 10.46 |
| 32 | mG*mA*mU*GAUCcP | B1:C7 | 2355.2701 | 1176.6278 | 11.72 |
| 33 | GUUAUCAACCP | G62:C70 | 2860.3635 | 1429.1745 | 12.06 |
| 34 | UUGAAAAAGcP | U71:G79 | 2947.4050 | 1472.6950 | 12.37 |
| 35 | UAAAAUAAGGCCP | U45:C55 | 3581.5019 | 1789.7437 | 15.09 |
| | (SEQ ID NO: 30) | | | | |
| 36 | AAAAUAAGGCUcP | A46:U56 | 3581.5019 | 1789.7437 | |
| | (SEQ ID NO: 31) | | | | |
| 37 | UAGAAAUAGCAAGUCP | U31:U44 | 4561.6230 | 1139.3980 | 17.98 |
| | (SEQ ID NO: 32) | | | | |
| 38 | AGAAAUAGCAAGUUCP | A32:U45 | 4561.6230 | 1139.3980 | |
| | (SEQ ID NO: 33) | | | | |
| 39 | AACUUGAAAAAGUGGCCP | A68:C83 | 5211.7159 | 1736.2313 | 18.57 |
| | (SEQ ID NO: 34) | | | | |

mG*, mA*, mU* represent 2′-O-methylguanosine, 2′-O-methyladenosine, and 2′-O-methyluridine with phosphorothioate linkers. ″cP″ represents cyclic phosphate.

These findings indicate that high pH-reaction conditions result in reduced production of short digestion fragments and increased production of long digestion fragments by Cusativin.

### Example 6: EDTA Modulates Ribonuclease Activity of Cusativin to Produce Longer Digestion Products

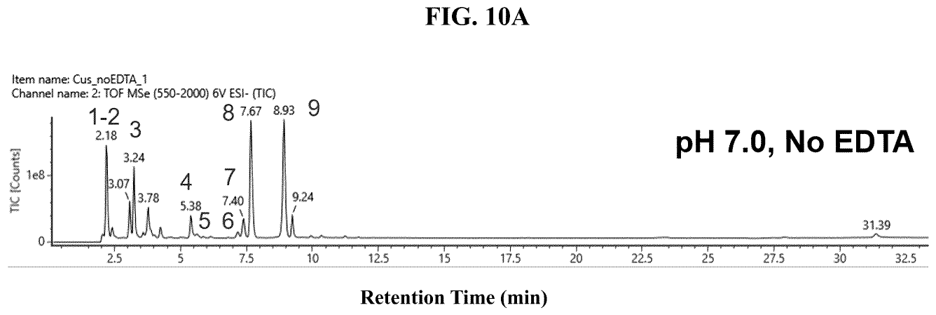

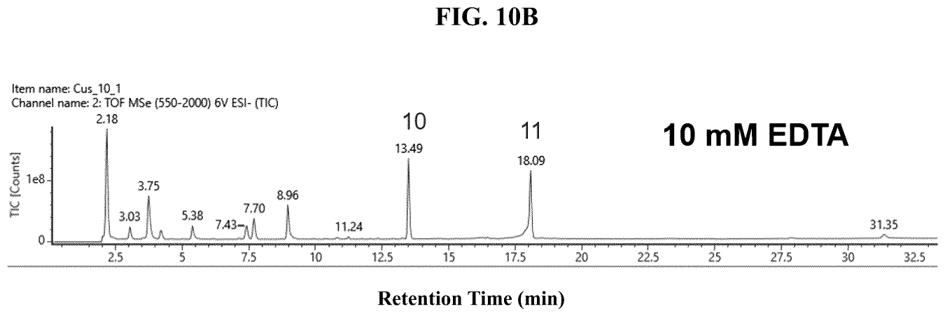

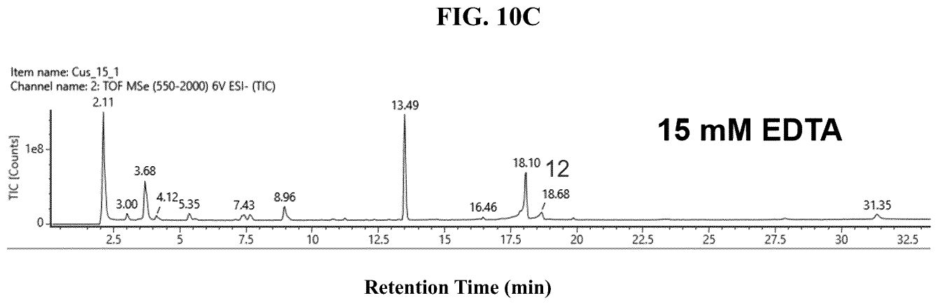

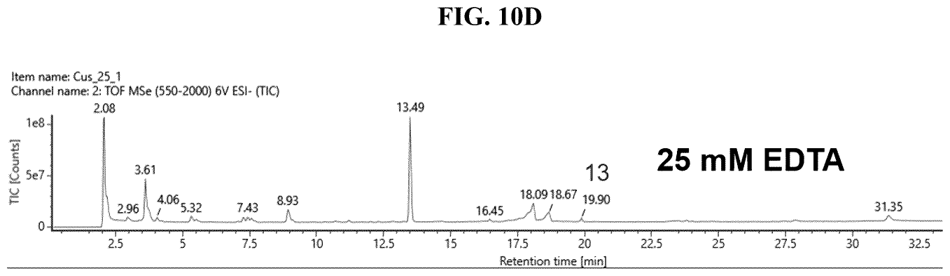

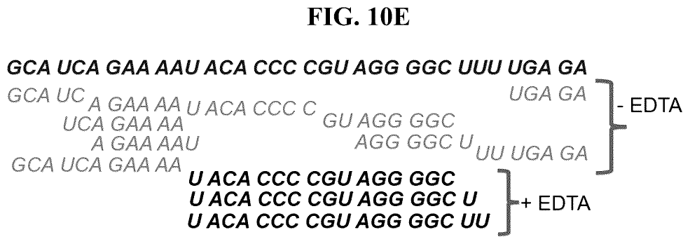

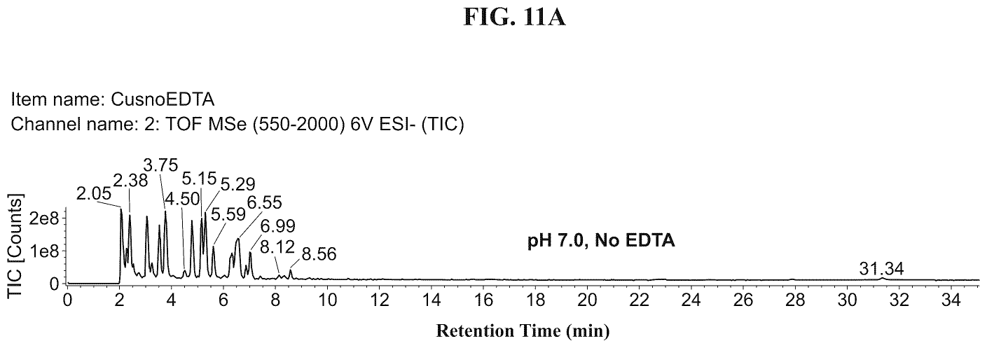

To determine the effect of EDTA on RNA cleavage activity of Cusativin, a 35-mer RNA polynucleotide was digested in the presence of 100 mM ammonium acetate (pH 6.5) without EDTA or with varying concentrations of EDTA (10 mM, 15 mM, or 25 mM) and subjected to IP-RP-LC-MS. Total ion chromatograms of 35-mer RNA digests performed without and with EDTA are shown in FIGS. 10A-10D. (FIG. 10A) No EDTA, (FIG. 10B) 10 mM, (FIG. 10C) 15 mM, and (FIG. 10D) 25 mM EDTA. A drop in intensities of peaks 4-9 (<10 min retention time) was observed as EDTA concentration increased compared to no EDTA samples. This was accompanied by the appearance of peaks 10-13 at elevated EDTA conditions. Table 10, below, shows a list of all oligonucleotide digestion products detected from the RNA digests along with their retention time, and FIG. 10E provides an overlay of predominately detected oligonucleotide digestion products to the nucleotide sequence of the 35-mer.

TABLE 10
Cusativin-generated oligonucleotide digestion products of a 35-mer RNA
polynucleotide in the presence and absence of EDTA

| Peak | Sequence | Retention Time (min) |
|---|---|---|
| 1 | GCAUC | 2.05 |
| 2 | UGAG-OH | 2.05 |
| 3 | UUUGAGA-OH | 3.68 |
| 4 | AGAAAA | 5.35 |
| 5 | AGGGGCU | 5.42 |
| 6 | AGAAAAU | 7.06 |
| 7 | GUAGGGGC | 7.36 |
| 8 | UACACCCC | 7.64 |
| 9 | UCAGAAAA | 8.93 |
| 10 | GCAUCAGAAAA | 13.47 |
| | (SEQ ID NO: 35) | |
| 11 | UACACCCCGUAGGGGC | 18.08 |
| | (SEQ ID NO: 36) | |
| 12 | UACACCCCGUAGGGGCU | 18.63 |
| | (SEQ ID NO: 37) | |
| 13 | UACACCCCGUAGGGGCUUU | 19.90 |
| | (SEQ ID NO: 38) | |

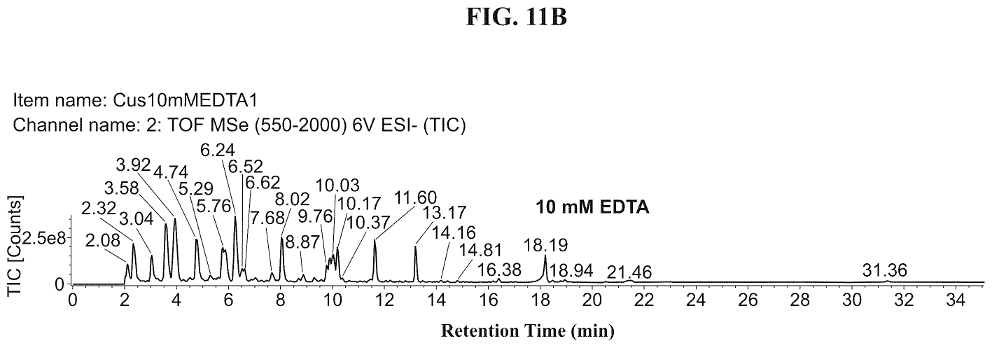

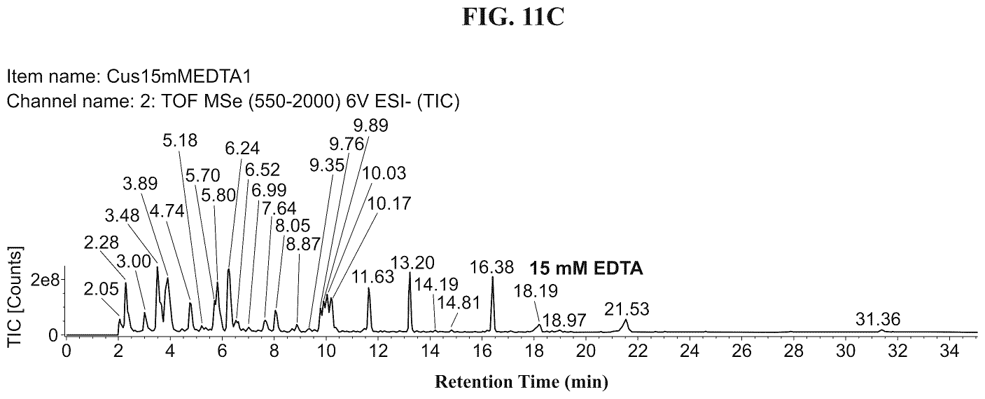

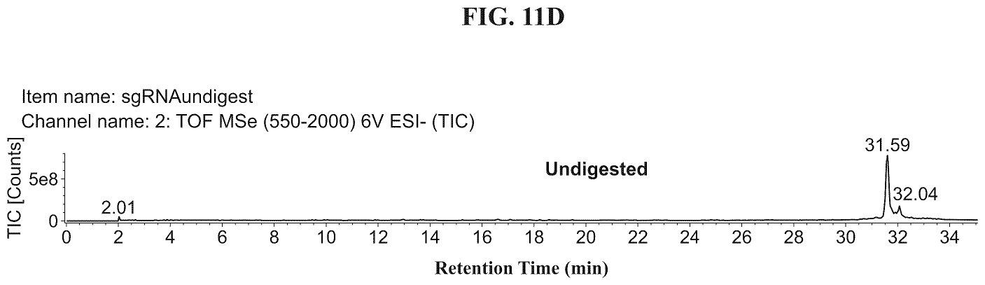

In a set of related experiments, Cusativin digestion of HPRT sgRNA was tested in the absence of EDTA or with varying concentrations of EDTA. HPRT sgRNA was digested in the presence of 100 mM ammonium acetate (pH 7.0) without EDTA or with 10 mM or 15 mM EDTA and subjected to IP-RP-LC-MS. Total ion chromatogram of sgRNA shows a change in oligonucleotide peak profiles between the EDTA-free conditions (FIG. 11A) and increased addition of EDTA to the digestion mix (FIGS. 11B-11C). In the absence of EDTA, smaller digestion products with shorter retention times (<9 min) were observed (FIG. 11A). As the EDTA concentration increased, peaks beyond 9 min retention emerged, representing longer digestion products (FIGS. 11B-11C).

Therse findings indicate that addition of EDTA to a Cusativin digestion reaction mixture results in reduced production of short RNA digestion fragments and increased production of long digestion fragments.

### Example 7: Thermal Modulation of Cusativin Enzymatic Activity

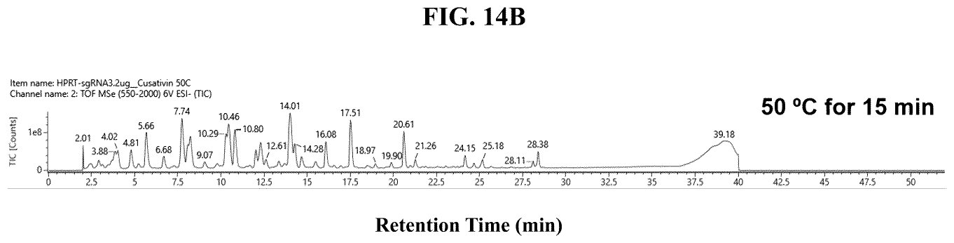

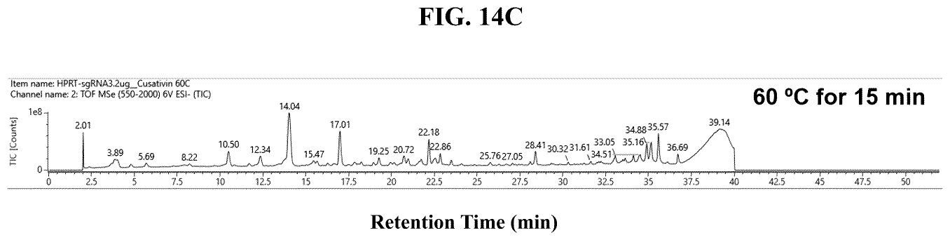

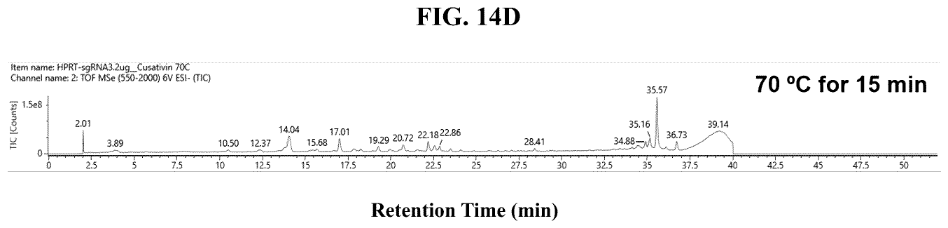

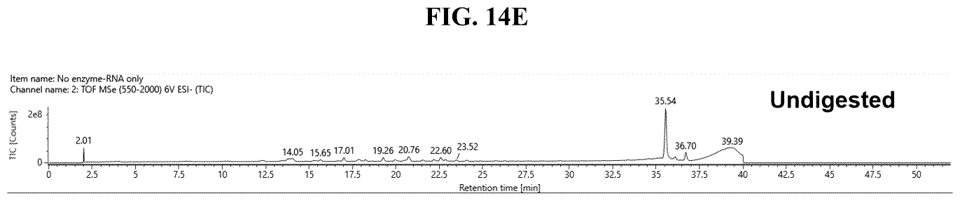

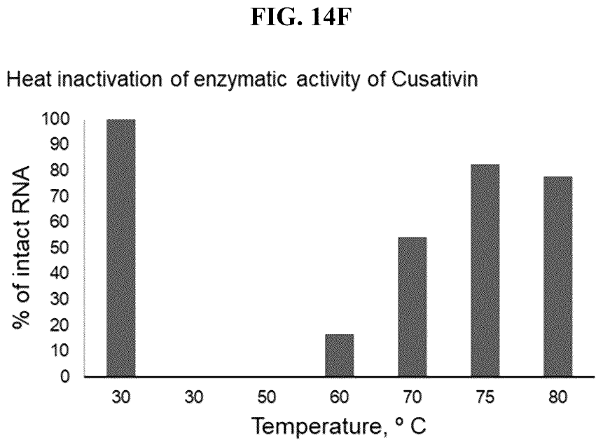

To determine the effect of reaction temperature on RNA digestion by Cusativin, an enzyme solution comprising HPRT sgRNA suspended in 200 mM ammonium acetate at pH of 9 was subjected to ambient temperature (FIG. 14A), 50° C. (FIG. 14B), 60° C. (FIG. 14C), 70° C. (FIG. 14D), 75° C. (FIG. 14F), or 80° C. (FIG. 14F) for 15 min followed by digestion of sgRNA at 30° C. for 1 hour. Prior exposure of Cusativin to 50° C. for 15 min had little to no impact on the RNA digestion patterns, as indicated by an identical oligonucleotide product profile to that of no exposure (FIG. 14A vs. FIG. 14B; see also FIG. 14F). Exposure to 60° C. for same duration modulated the TIC profile (FIGS. 14C, 14F), where some digestion products with shorter retention time (<12.5 min) exhibited decreased abundance. Moreover, an increased abundance and appearance of products with longer retention time (>12.5 min) was observed, indicating decreased activity of the enzyme. However, exposure to 70° C. did not result in digestion of sgRNA, as indicated by a clear peak of intact RNA at 35.97 min (FIGS. 14D, 14F). A small change in peak area was observed for Cusativin enzyme exposed to 75° C. or 80° C. (FIG. 14F). These results indicate that Cusativin exhibits a temperature-dependent inactivation that becomes readily apparent at 70° C.

### Example 8: Further Evaluation of pH-Dependence of M1 Enzyme Activity

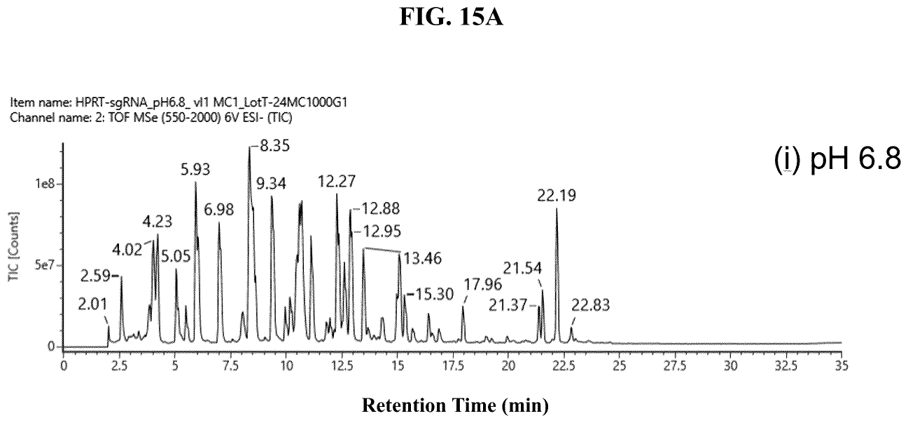

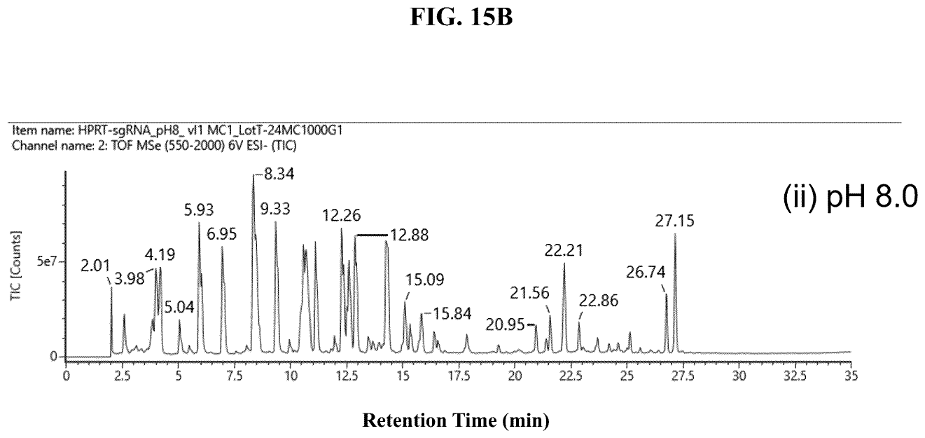

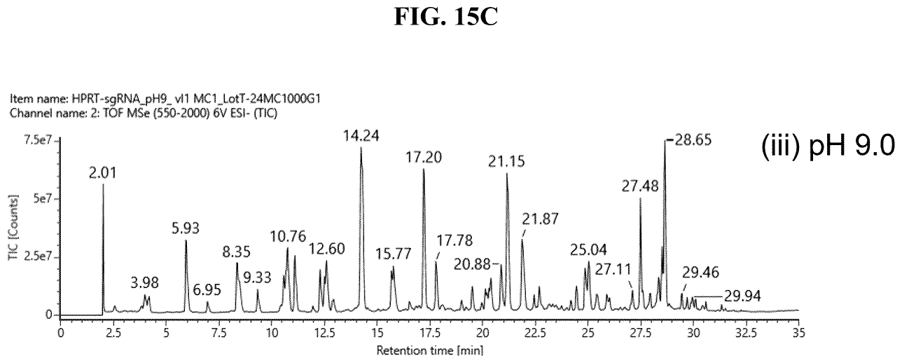

The effect of pH on RNase MC1 activity was further explored. HPRT sgRNA was digested with RapiZyme MC1 in 100 mM ammonium acetate buffer at pH 6.8, pH 8.0, pH 9.0 for 1 h at 30° C. The MC1 enzymes were heat inactivated at 70° C. for 15 min. Resulting digestion products were analyzed by IP-RP-LC-MS. At lower pH (6.8) medium (RT=˜22 min) to smaller digestion products were observed (FIG. 15A). As the pH increased, digestion product length increased (RT=28.65 min at pH 9.0; FIGS. 15B, 15C). Smaller digestion products (up to RT=13.46 min) decreased considerably at higher pH (FIGS. 15B, 15C). These findings indicate that oligonucleotide or polynucleotide digestion reactions with MI enzyme in which larger digestion products are desired may benefit from use of high pH values (e.g., pH 9). Digestion at a pH 8 may be optimal for digestion reactions in which a wide range of digestion product lengths is preferred.

### Example 9: Further Evaluation of pH-Dependence of Cusativin Enzyme Activity

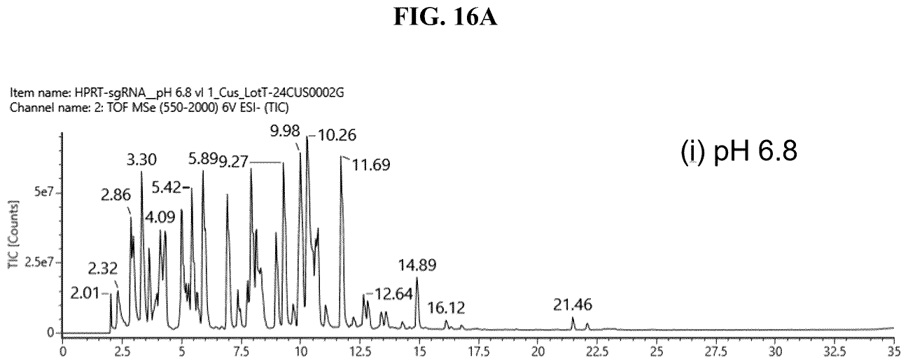

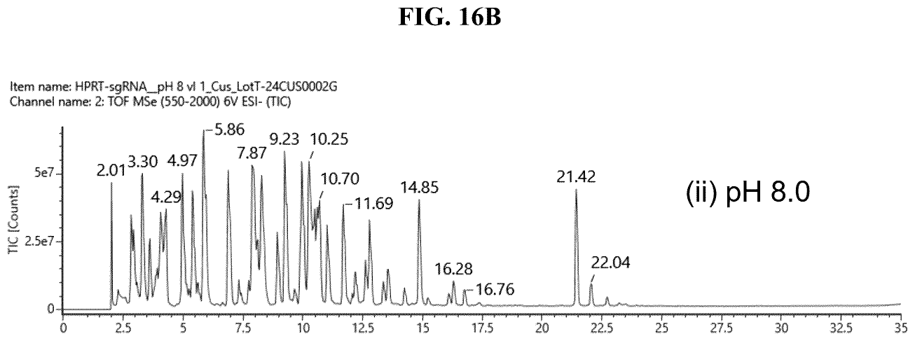

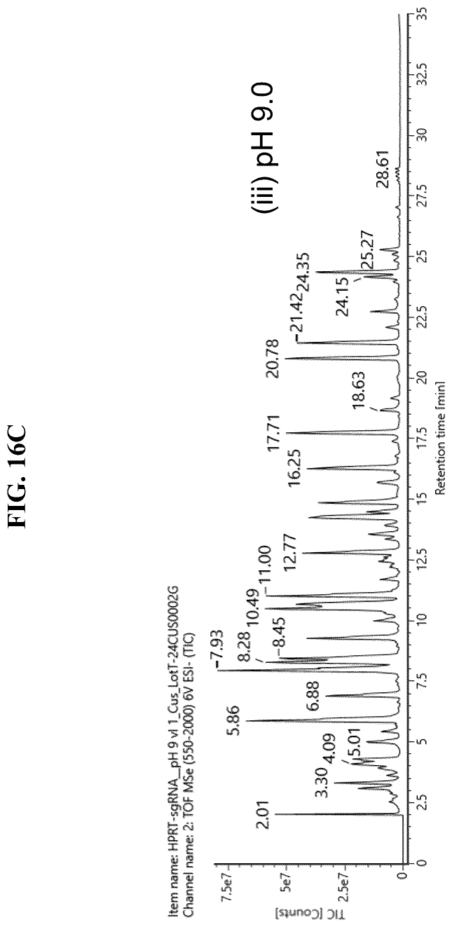

The effect of pH on RNase Cusativin activity was further explored. HPRT sgRNA was digested with RapiZyme Cusativin in 100 mM ammonium acetate buffer at pH 6.8, pH 8.0, pH 9.0 for 1 h at 30° C. The Cusativin enzymes were heat inactivated at 75° C. for 15 min. Resulting digestion products were analyzed by IP-RP-LC-MS. At lower pH (6.8), medium (RT=˜21 min) to smaller digestion products were observed (FIG. 16A). As the pH increased, digestion product length increased (RT=25.27, 28.65 min at pH 9.0; FIGS. 16B, 16C). Smaller digestion products (up to RT=13.46 min) decreased considerably at higher pH (FIGS. 16B, 16C). These findings indicate that oligonucleotide or polynucleotide digestion reactions with Cusativin enzyme in which larger digestion products are desired may benefit from use of high pH values (e.g., pH 9).

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

1. A method of inducing site specific cleavage of one or more RNA polynucleotides, comprising contacting the one or more RNA polynucleotides with an RNase MC1 (MC1) enzyme in a solution having a pH of 6.5-9.5.

2. The method of claim 1, wherein the solution has a pH of 8.0.

3. The method of claim 1, wherein the solution has a pH of 9.0.

4.-7. (canceled)

8. A method of inducing site specific cleavage of one or more RNA polynucleotides, comprising contacting the one or more RNA polynucleotides with a Cusativin enzyme in a solution having a pH of 6.5-9.5.

9. The method of claim 8, wherein the solution has a pH of 9.

10.-13. (canceled)

14. The method of claim 1, further comprising the step of inactivating the enzyme at a temperature of 70° C.-80° C.

15. The method of claim 1, wherein the method produces one or more RNA digestion products having a length of at least 3 nucleotides.

16. The method of claim 1, wherein the method produces one or more RNA digestion products having a length of between 3 and 30 nucleotides from an RNA polynucleotide having a length of 30-100 nucleotides.

17. (canceled)

18. The method of claim 1, wherein the method is used in conjunction with a method for sequencing the one or more RNA polynucleotides.

19. The method of claim 1, wherein the method is used in conjunction with liquid chromatography-mass spectroscopy (LC-MS) analysis of the one or more RNA polynucleotides.

20. The method of claim 19, wherein the LC-MS is ion pairing reversed phase LC-MS (IP-RP-LC-MS).

21. The method of claim 1, wherein the solution further comprises ethylenediaminetetraacetic acid (EDTA) at a concentration that is between 2 mM and 5 mM.

22. (canceled)

23. The method of claim 8, wherein the solution further comprises EDTA at a concentration that is between 10 mM and 15 mM.

24.-28. (canceled)

29. A method of determining the nucleic acid sequence of an RNA polynucleotide of interest, comprising:
(a) contacting the RNA polynucleotide of interest with an MC1 enzyme in a solution having a pH of 6.5-9.5 to produce a population of RNA oligonucleotides, wherein at least two RNA oligonucleotides within the population have a nucleic acid sequence overlap of at least 1 nucleotide;
(b) subjecting the RNA oligonucleotides to LC-MS analysis to determine the nucleic acid sequence of each of the RNA oligonucleotides within the population; and
(c) aligning the nucleic acid sequences of the plurality of RNA oligonucleotides using the overlap between the at least two RNA oligonucleotides, thereby determining the nucleic acid sequence of the RNA polynucleotide of interest.

30. The method of claim 29, wherein the solution has a pH of 8.0.

31. The method of claim 29, wherein the solution has a pH of 9.0.

32. A method of determining the nucleic acid sequence of an RNA polynucleotide of interest, comprising:
(a) contacting the RNA polynucleotide of interest with Cusativin enzyme in a solution having a pH of 6.5-9.5 to produce a population of RNA oligonucleotides, wherein at least two RNA oligonucleotides within the population have a nucleic acid sequence overlap of at least 1 nucleotide;
(b) subjecting the RNA oligonucleotides to LC-MS analysis to determine the nucleic acid sequence of each of the RNA oligonucleotides within the population; and
(c) aligning the nucleic acid sequences of the plurality of RNA oligonucleotides using the overlap between the at least two RNA oligonucleotides, thereby determining the nucleic acid sequence of the RNA polynucleotide of interest.

33. The method of claim 32, wherein the solution has a pH of 9.0.

34. The method of claim 29, wherein the nucleic acid sequence of each RNA oligonucleotide within the population is determined using a unique mass tag of each ribonucleotide within the RNA oligonucleotide.

35. (canceled)

36. The method of claim 29, wherein step (a) further comprises a step of inactivating the enzyme at a temperature of at least 70° C.


SEQUENCE LISTING (39 sequences)

SEQ ID NO: 1 - protein, 190 aa, synthetic construct
FDSFWFVQQWPPAVCSFQKSGSCPGSGLRTFTIHGLWPQQSGTSLTNCPGSPFDITKISHLQSQLNTLWPNVLRANNQQFWSHEWTKHGTCSESTFNQAAYFKLAVDMRNNYDIIGALRPHAAGPNGRTKSRQAIKGFLKAKFGKFPGLRCRTDPQTKVSYLVQVVACFAQDGSTLIDCTRDTCGANFIF

SEQ ID NO: 2 - DNA, 570 bp, synthetic construct
  source 1..570: mol_type = other DNA
ttcgactccttttggtttgttcagcagtggccgccagcagtttgcagtttccaaaaaagtggtagttgccctggtagtggtctgcgcacgtttactatccatggcctgtggccgcaacagagcggcacaagcctgactaactgtccgggttctccgtttgatatcaccaagatcagccacctgcagagccagctcaataccctgtggccgaacgtgctgcgtgccaataaccaacagttctggagtcacgagtggacgaaacatggcacatgctccgaaagcacctttaaccaagcggcctacttcaaacttgcggtcgacatgcgcaacaactatgatatcatcggggccttgcgtccgcacgcagcaggcccgaacggccgtaccaaatcacgtcaggccatcaaagggtttctgaaagcgaaatttggtaaatttccgggcttacgttgtcgtaccgatccgcaaacgaaagttagctatctggttcaagtcgtcgcatgcttcgcccaggatggttcaaccttaattgattgtacccgcgacacgtgtggcgccaatttcatcttt

SEQ ID NO: 3 - protein, 217 aa, synthetic construct
MEKTKSVDVVFFVFVLTILFPIVKSQTFDDFWFVQQWPPAVCTLQSGRCVGRGTRSFTIHGLWPQKGGRSVTNCTGNQFDFTKIAHLENDLNVVWPNVVTGNNKFFWGHEWNKHGICSESKFDEAKYFQTAINMRHGIDLLSVLRTGGVGPNGASKAKQRVETAISSHFGKDPILRCKKASNGQVLLTEIVMCFDDDGVTLINCNKARSNCAGSFIF

SEQ ID NO: 4 - DNA, 651 bp, synthetic construct
  source 1..651: mol_type = other DNA
atggaaaaaacaaagagtgtcgatgttgtgttcttcgtgtttgtcttaaccattttgtttcctatcgttaaatcgcaaactttcgatgatttttggtttgttcagcaatggccaccagctgtttgtactttacaatcaggacgatgtgtaggacgagggactcgatccttcacaattcatggtttgtggcctcaaaagggaggacgttctgtgactaattgtactggcaatcaatttgatttcactaagattgcacatttagaaaacgatctaaacgtagtttggccgaatgtggtgacgggaaacaacaaattcttttggggtcatgaatggaacaaacatgggatttgctcagagagcaagtttgacgaagcgaagtacttccaaacagcgataaacatgaggcacggtatcgaccttcttagtgtgctgagaactggtggagtaggaccaaatggagcctccaaggcaaagcaaagagtcgaaaccgcgatttcatcccattttggaaaagatccaattcttcgatgtaaaaaagcatcaaacggccaagtcttattgacggagattgtgatgtgcttcgatgacgatggtgtcacccttataaactgtaacaaagcaagatccaattgtgctgggagctttattttt

SEQ ID NO: 5 - DNA, 651 bp, synthetic construct
  source 1..651: mol_type = other DNA
atggaaaagaccaaaagtgtcgatgtggtgttttttgtatttgtcctgactatcctgttcccgatcgtcaagagtcagacatttgacgatttctggtttgttcaacaatggcctccggcggtttgtacgttacagtctggccgctgtgttggtcgtggaactcgctcatttactattcatggcctgtggccacagaaaggcggccgtagtgttacgaactgtactggtaaccagtttgattttacgaaaattgcacacctggaaaacgatttgaacgttgtatggcccaatgtggtcacgggcaataacaaattcttttgggggcatgagtggaataagcacggcatctgttcagaatccaaatttgatgaagccaaatatttccagaccgcgattaacatgcgccatggcatcgatctgttgagcgtcctccgtaccggcggtgttggtccaaatggcgctagcaaagccaaacaacgcgtggaaaccgccatctcctcccactttggtaaggatccgattctgcggtgcaaaaaggcgtcgaacggccaagtgctgctgacggagattgtgatgtgctttgatgacgacggcgtcaccctgatcaattgtaataaggcgcgctccaattgcgcaggctcatttattttt

SEQ ID NO: 6 - RNA, 11 nt, synthetic construct
  source 1..11: mol_type = other RNA
gcatcagaaaa

SEQ ID NO: 7 - RNA, 16 nt, synthetic construct
  source 1..16: mol_type = other RNA
tacaccccgtaggggc

SEQ ID NO: 8 - RNA, 17 nt, synthetic construct
  source 1..17: mol_type = other RNA
tacaccccgtaggggct

SEQ ID NO: 9 - RNA, 18 nt, synthetic construct
  source 1..18: mol_type = other RNA
tacaccccgtaggggctt

SEQ ID NO: 10 - RNA, 100 nt, synthetic construct
  source 1..100: mol_type = other RNA
  modified_base 1..3: mod_base = OTHER; note = 2'O-methyl ribonucleotide
  modified_base 1..4: mod_base = OTHER; note = phosphorothioate linkage
  modified_base 97..99: mod_base = OTHER; note = 2'O-methyl ribonucleotide
  modified_base 97..100: mod_base = OTHER; note = phosphorothioate linkage
gatgatctctcaactttaacgttttagagctagaaatagcaagttaaaataaggctagtccgttatcaacttgaaaaagtggcaccgagtcggtgctttt

SEQ ID NO: 11 - RNA, 10 nt, synthetic construct
  source 1..10: mol_type = other RNA
tttaacgttt

SEQ ID NO: 12 - RNA, 10 nt, synthetic construct
  source 1..10: mol_type = other RNA
tagtccgtta

SEQ ID NO: 13 - RNA, 11 nt, synthetic construct
  source 1..11: mol_type = other RNA
taaaataaggc

SEQ ID NO: 14 - RNA, 12 nt, synthetic construct
  source 1..12: mol_type = other RNA
tagagctagaaa

SEQ ID NO: 15 - RNA, 13 nt, synthetic construct
  source 1..13: mol_type = other RNA
accgagtcggtgc

SEQ ID NO: 16 - RNA, 14 nt, synthetic construct
  source 1..14: mol_type = other RNA
taaggctagtccgt

SEQ ID NO: 17 - RNA, 13 nt, synthetic construct
  source 1..13: mol_type = other RNA
tagcaagttaaaa

SEQ ID NO: 18 - RNA, 14 nt, synthetic construct
  source 1..14: mol_type = other RNA
tagaaatagcaagt

SEQ ID NO: 19 - RNA, 16 nt, synthetic construct
  source 1..16: mol_type = other RNA
taaggctagtccgtta

SEQ ID NO: 20 - RNA, 25 nt, synthetic construct
  source 1..25: mol_type = other RNA
tgaaaaagtggcaccgagtcggtgc

SEQ ID NO: 21 - RNA, 26 nt, synthetic construct
  source 1..26: mol_type = other RNA
ttgaaaaagtggcaccgagtcggtgc

SEQ ID NO: 22 - RNA, 11 nt, synthetic construct
  source 1..11: mol_type = other RNA
gcatcagaaaa

SEQ ID NO: 23 - RNA, 16 nt, synthetic construct
  source 1..16: mol_type = other RNA
tacaccccgtaggggc

SEQ ID NO: 24 - RNA, 17 nt, synthetic construct
  source 1..17: mol_type = other RNA
tacaccccgtaggggct

SEQ ID NO: 25 - RNA, 18 nt, synthetic construct
  source 1..18: mol_type = other RNA
tacaccccgtaggggctt

SEQ ID NO: 26 - RNA, 11 nt, synthetic construct
  source 1..11: mol_type = other RNA
taaaataaggc

SEQ ID NO: 27 - RNA, 11 nt, synthetic construct
  source 1..11: mol_type = other RNA
gcatcagaaaa

SEQ ID NO: 28 - RNA, 16 nt, synthetic construct
  source 1..16: mol_type = other RNA
tacaccccgtaggggc

SEQ ID NO: 29 - RNA, 17 nt, synthetic construct
  source 1..17: mol_type = other RNA
tacaccccgtaggggct

SEQ ID NO: 30 - RNA, 11 nt, synthetic construct
  source 1..11: mol_type = other RNA
taaaataaggc

SEQ ID NO: 31 - RNA, 11 nt, synthetic construct
  source 1..11: mol_type = other RNA
aaaataaggct

SEQ ID NO: 32 - RNA, 14 nt, synthetic construct
  source 1..14: mol_type = other RNA
tagaaatagcaagt

SEQ ID NO: 33 - RNA, 14 nt, synthetic construct
  source 1..14: mol_type = other RNA
agaaatagcaagtt

SEQ ID NO: 34 - RNA, 16 nt, synthetic construct
  source 1..16: mol_type = other RNA
aacttgaaaaagtggc

SEQ ID NO: 35 - RNA, 11 nt, synthetic construct
  source 1..11: mol_type = other RNA
gcatcagaaaa

SEQ ID NO: 36 - RNA, 16 nt, synthetic construct
  source 1..16: mol_type = other RNA
tacaccccgtaggggc

SEQ ID NO: 37 - RNA, 17 nt, synthetic construct
  source 1..17: mol_type = other RNA
tacaccccgtaggggct

SEQ ID NO: 38 - RNA, 19 nt, synthetic construct
  source 1..19: mol_type = other RNA
tacaccccgtaggggcttt

SEQ ID NO: 39 - RNA, 35 nt, synthetic construct
  source 1..35: mol_type = other RNA
gcatcagaaaatacaccccgtaggggcttttgaga